SEQUENCE LISTING (18 sequences)

SEQ ID NO: 1 - DNA, 3001 bp, Aspergillus niger
ggttataattcaaaattcaacttccacctttgtttcaccggcggccacggcattcctgcatgactaacgttctgtaaatggacccgataacacccagcacgttgcagcagagaaggtactctctcacacgcactgctctttatagttgccgagacggccgccgaggagaaaaccgccggcctgtggccactattcgctggaaggaaccctgccagtcgaacacacccgcccgtgatcgccaggggccgatggatttccccccgcatccttgtcggttcatgagtgaagactttaaatcccatctagctgacggtcgggtacatcaataactggcagcctagtttccaagacacggagaagcatgtaatcgctatttatagaatgctgggatcggacccgtcgaatggtcttccgatgggaagtgacaactcacattgtcatgttggccttactcaatccaacgggatctgacctgctttggctaacctagtataaatcagcatgtctctcctttgatacatcggatcgttcctcaaatatagttatatcttcgaaaaattgacaagaaggatgacaatctttctgtttctggccattttcgtggctacagctctggcagccacgcctgcagaatggcgctcccagtcgatatatttcctgctcaccgatcgctttgcgcgaacggataattctaccactgcttcttgtgacttgagcgctcgggttagtcacagcatgttctagaatctccaattgattcgctgacagatctagcaatattgcggtggatcctggcagggcatcatcaatcaggtcggtccgtccatcgttgcagcactatctacatcaacgtttgtttggcaaattaacatccattagctggactatattcaaggaatgggctttacagcgatctggatcacacccgtaactgcacagatcccccaagatactggttacggacaggcatatcacggatactggcagcaggacgcgtgagatgctacctctatcgcccggatgaatgtatatccttcttaccatgcagacagttatgccctgaactcccattatggtacggcagacgatctcaaagctctggcttcagctcttcactcacggggcatgtatctcatggtggacgttgttgccaatcacatggtatgttcttagcctcccacgggaccttagctttatatctgacagcgatagggccacaatggtacggggagctctgtggactacagtgtttataggccatttaattcgcaaaagtactttcacaacctctgttggatctctgattacaataaccagacaaacgttgaagactgctggctaggcgataacaccgttgccttgccggatcttgatactaccagtacggaggtgaagaatatgtggtatgactgggtcgagtctctcgtctctaactactccggtaatcctacctttacttcgctattttctgcctcttatgagacaaagactaacaaatatcaagtcgacggcctccgcgtagacacagtcaagaacgtacagaagaacttctggcccggctacaacaatgcttcaggcgtgtactgtattggagaagtcttcgatggggacgcctcatacacctgtccttatcaggaagacttggacggagtccttaattaccccatgtaagccctacatttaaccccattgaatgcttgccaacgacttgcaataggtactatccactcctccgggctttcgaatccaccaacggcagtatcagcgacctctataacatgatcaacaccgtgaaatccacctgcagagattctacgcttctagggaccttcgtcgaaaaccacgataacccacgctttgccaagtaagaatatcctctccgagttcaccattacaaacacaagagctcacctcagaagctacacaagcgacatgtccctagccaaaaatgccgcaacattcactatcctggctgacggcattcccatcatatacgccggtcaggaacagcactatagcggcggtaatgacccctacaaccgcgaagcgacctggctttcaggctacaagaccaccagcgagctctacacccatatcgccgcatcgaacaagattcgcacccacgctataaaacaggataccggatatctcacctacaaagtaatcttcattcgagtccatgtgtggtacaatctatctgactagaacctattctagaactaccccatctaccaagacacctcgacccttgccatgcgcaaaggctacaatggcacccaaactatcacggtcctttctaaccttggcgcctcggggtcctcatacacactctccctcccaggaacaggctacacagccggccaaaagattactgaaatctatacctgcacgaatctaacagtcaactcaaatggctcggtgccagtacccatgaagagcgggttaccgcggatcctctatcctgcagataagttggttaatggaagctcattttgcagttagttccctgcctatatgtttcaataagccgtatttgcatgcgcgtccatcgcattgattctttatggattgatcaactgatacattcgcacaggctctgcgatcactgttttcaaggatgcaggaggaggcgtgttgttctttagctacactgtgtaagtgtatcagatcatatcatggccactagacgatgacccctgtgatcgtgatccagatgctgacttcaatttgtagaattttcgctcaggtgcttattgctataatgacgtgattaggatgtctcagatgacagtatatattattaggtgacgtgatgattctatgtgtaatggattcttcaagatcattttatattattagtatccacagtattcatttgcctgcaaagtaagtatggataaaatcactaccaccgacgtagtagttgtacctagtccttagtgtctcaaagccacagtcactacaacctcacgaaacgacggagtaaacttatttctattga

SEQ ID NO: 2 - DNA, 2511 bp, Aspergillus niger
catgaacgctttcccagccgaaatgtgggaagttaatttgccttgtccacttcgccctccagcgcagccctgccctcacgacaatcagtcaatctcccgtgacaatcccaggggatcttcgtgttctaacgctgctacccgttctcgaatgttcgggtgaactggagtactagagtagattaccatcgagcgttacaaatttagatggcgtctttatttttggtccgcgttgagggtcgttgaaagtgatggatcagggcttcgggcacgcgatgcgaacgagggtgggggagagactacctatttatggtacctccggcttctaccggccttgtctgcagccccccggtctggcgctcttatctctcttgctaagttgctatagctatcgggggtccgccccccctgaatcgcgttgccttgacttgtcattctttcttttccccgttattgcaacgggaacagcgcacccggcctcaaacatcatgctcagcggcccgtttggctgaatcgcgtgaaattgaaacgcttaaaacacattcgttagagtaagctgttgcttgtcataattcattcggtcacccaagcccacccacgggaagcagaacctccacaagggggcacagagggttgatcgagggtagtactgccaatgatgtttcgaaaatccgcttccctcctgggccaacggctcatggccgtttgtctcctgtgctggtgcgtttcgctagcgaccgccgcaagcacagaagaatggaagacgcgatccatctaccagacgatgacggatcggtttgccctcaccaacggttcgacgaccgcaccatgcaatactacggtagccaactactgtggcgggtcttggcaggggacgatcgataagctggactacatccaaggcatggggttcgatgcgatcatgatctctcccgtaattaaaaacattgcggggcgatctaaggacggtgaggcctaccatggatactggcctctggatctatacgagatcaattctcatttcggaacccgggaggaactgttgaagctgagcgaggagatccacgcacgcggcatgtacttgctgctggacgtcgtcatcaataatatggcttacatgacggacggcgaggatcctgcgacgaccattgactacaatgttttccctcagttcaatggatcttcatacttccacccctactgtcttattacgaactggaataactatacggacgcgcagtggtgtcagactggtgacaattatactgcactcccagatctgtacacagaacacaccgcggtgcagaacatcttaatggactggagtaaatcggtcatcagcaattactccgtcgacgggctacgaatcgacgctgccaagtccctcactcccagctttctgcctacatatgcgagtaccgtgggcggattcatgaccggcgaagtcatggattcgaacgccaccaacgtgtgcaaatatcagacggattacctgccaagtcttccaaactaccccctctattactccatgatcacggccttcctcaacggcgagcctgcgaccttgctcgaggaaatcgcgacgatcaatgatctttgccctgatacgttcgcaatggtcaacttcatcgaagatcaagacgttgaccgatgggcctacatgaatgacgacatcatgctagctaaaactgcactgaccttcatgatgctctacgacggtattcccttggtctaccagggtctggagcaggccattgcctattccaaccgagcggccttgtggttgacagatttcgacaccaatgcgacgctttataaacacatcaagaaactcaatgccatccgcaaacatgccattaaccttgattccagctacatcagttcgaaaacatatcccatctatcaaggaggtagcgagttggctttctggaagggcaacaatggacgccaagtcatcatggttttatctacggcgggctcgaatggttcagcttatactctgacactacccgtaagctatggggctagtgaggtggtcacggaagtgctgaactgcgtcaattatacggtcaacacttacagtcaattggtcgtggacatggataagggcgaacctcgggtcttctttcccgcttcgatgatgccggggagcgggctgtgtggctacaatacttctaatgtgacgtattctgagctgagacttgctgctgtgggatcttcgtcgtctgctggtagccactctgtcataccgtctgcttttgcttcgcttttcatggcgatagttgcatttttggcattccggatataagttcatattttttctactcagttttataccagcaaataaaacgatgtcctatgaatatacacatttcaaaagtggagacaaaattatatctttatatccctagcacctccctttccgtatcctttcctgcaataaagacctattcagcattgggagcagaattacgaagcacagtctcctcagttcgcatacccgcccacctccaa

SEQ ID NO: 3 - DNA, 2501 bp, Aspergillus niger
ccccagcattccacttcttctcgcttcccctcttctctctcctctccctctcctctcctctcctttcccttcccttcccttccttcctcctcactcattctttcgctctcgtcacatcgaccaatgcatccgtcactgaagtgaccccctcttcctgggctgttcccttgccattcatttttttctttggatctgaccattgccattctttctctacaatctggtcccacttctttcgttcactctttcatttcgtgcctagcccgtatccttccttttagcgtaaccggctatctcccagcattcgacagtcaccttatcgtatctccaagtcgggccccgcaattgctcggcaggcacatgtacgcaacccttgatcattaagataattccaatctcccagtgactgtagccatggtctcaatgtcggccctgcggcacgggcttggggtcctctatcttgctagctggctggggtcgtcgctggctgccagcaccgagcaatggaagtcccggtcaatctatcagaccatgacggatcggttcgcgcgcactgatggctcaaccacctccccctgcaacaccacggagggtctgtactgtggtggtacctggcgcggcatgatcaaccatctggattacatccaggggatgggctttgatgccgtcatgatctcccctatcatcgagaacgtcgaaggtcgcgtggagtacggagaagcctatcacggctattggcccgtggatctgtactccctcaactcgcactttggaacccaccaggatctactggacctgagtgatgccctgcatgctcgcgacatgtatctgatgatggacacagtcatcaacaacatggcctacatcaccaatggctccgaccccgccacccacatcgactactcaaccctgacccccttcaatagctcgtcttactaccatccctactgtaagatcaccgactggaataacttcaccaatgcccagctgtgccaaaccggtgacaacatcgtggccttgcccgatctgtataccgagcatgccgaggtgcaagaaaccctgagcaactgggccaaggaagtcatctccacctattccatcgacggcctccgcatcgatgccgcaaaacatgtgaaccccggcttcctgaagaatttcggcgatgcgcttgatatcttcatgaccggcgaagtgctgcagcaagaggtcagcacaatctgcgattatcagaacaactacatcggcagtctcccgaattaccccgtctactacgccatgttgaaggcctttaccttgggcaacaccagcgccctggccactcaggtccagtcgatgaagaattcctgtaatgatgtgaccgccttgtcgtcattctccgaaaaccacgatgtcgcacgatttgccagcatgacccatgacatggctgtatgaactctgcttgactttacagacccctttcctagctaacattgtccctactagctcgcgaagaatattctcacattcactcttctcttcgacggtgttcccatgatctatcaaggtcaagagcagcatctcgacggccctggcagtccggaaaaccgagaagcgatatggctctctgagtacaacaccgacgccgagctctacaagttgatcggcaagttgaacgccatccggaagcacgcctaccgactcgacaatcactaccccgatgtcgagacgtaccccatcttcgaaggtggcagtgaactgggattccgcaagggaatcgaaggtcgacaggtggtcatgcttctgtccacgcaaggcacgaacagcagcgcttacaacctctcgatgccggtgagtttcacgggtggcacagtagtgaccgagattctgaactgcgtcaactacacggtcaacacccagagcgaactcgtggttccgatggataaaggagagccgcgcgtgttcttcccggcggatctgatgcccggcagtgggctgtgcggacttcctgtcgcgaatgtcacctatgctgccttgaggacgcagggtgcagcggcggcggaggctgccttgtcgctaggtatcaagactgatgcagcttctagtgctttgctttctctggggctgtctgtggtggcgggtctgattgtggggatgtggtaatatgctgatagctacagatgggttgagatatattatcatgtttcagtgttctttgtatgtatttgcagtaacgatacccttacctagtatattgtatatattcacttcgctatatccactactactcgcagatactagccgcatctcgctttattccacactagttactagcacttcagcactaagcaataaacagaacgaagacaaattactagtccagtatatatataaatagagatcacctgtaaattcttcagaaaacccaaacgggtacctgcatccaacagctgttgacgatcatggctggctgatcctctggaggcaaatccgttgggcctcggtagctttccgaggaaaccgcc

SEQ ID NO: 4 - DNA, 3080 bp, Aspergillus niger
catctcgctttattccacactagttactagcacttcagcactaagcaataaacagaacgaagacaaattactagtccagtatatatataaatagagatcacctgtaaattcttcagaaaacccaaacgggtacctgcatccaacagctgttgacgatcatggctggctgatcctctggaggcaaatccgttgggcctcggtagctttccgaggaaaccgcccgagctaaaaatactcgctcatcagccagacaggcccactttaaggacccattgaaaattgaaatgaagtcatgcacaccgtgttaactagctaactagctagttcttcttagtgtcaagacagcagctattctaacgttcttccgtgcttacaccacagcccagctccgtcgctgcattcgactcttcaccttccatccatcatccatccattctacatggacacagacagtaaacctagctaatccctcctccgcaccgcagtaaaaaaatcctcacctcccgaaccaccaacgacctacaaacataacccaagaaaaaaagaatacaagcaaaatgttcagtttcctcccctgcttcaaaactcgccgacaacgcaccaaatcccaaactcaatcccaaaagcaaatagagggttagccaaacacccccaactacccataccaagtcaattcaattcaaatcatagtaacaaattcaacctccccaatagaaaaagccaacgccatagaatccctcccctcatggcactcccccacggaaaacaccctcctatttcaagccttcgaatggcacgtccccgcaacccccaacaccgcggacaagcgcagccactggcgccggctccagcatgccttaccagctatccactccctcggtgtaaccagcatctggataccgcctggatgcaagggcatggacacaaatggaaatgggtatgatatctacgatttatatgatctaggcgaattcgaccagaaaggagcagtgcgaacgaaatggggcacgcgcggagaattagaggatcttgttcgagatgcaaatgctctgggcgtaggtgtattatgggatgcggtgttgaatcataaggctggggcggatagcgtggagaggttcgagggtgtgagggttgatgatgatcgtatatacccctttcccatctctccgttcttctatgtatagtgtgtatattgacaaatgcttcacaggacgagatatcgaagatggtaatccccaacaaatctccggctggacgtccttcaccttccccggccgcggcaccacatacagcccccttcaataccactggcagcacttcagcggcgtcgactgggacgacgcccaacaacgcaaagcaatctacaagatcctcgacccttcccgccccgacaagaactgggcccaggacgtcggcacagacgagaacggcaactatgactacctgatgttcgcggacctggacttctcacacccggaggtccgggaggatgtactgcgctgggggaagtggattatgtctgtgttgccgttgagcggaatgcggttagatgcggcgaagcatttctcgacggcatttcagagggactttattgattgtgtgcggcaggaggccggggacaggaaggtatttgtgattggggagtattggagtggggagttaagggcgctgcttaggtatctggaggagatggagtatcgggtggcggcggtggatgtgccgctggtagagaggttctcgaggttgtcgagggtgaagagggcggatttgaggggagtgttgagggggacgttggtggagagcaggccggggaatgcgttggtgagtttatcccagtgatccaattcgggggtggtgaggctgatgtgtgtgtggtgtagacattcgttacgaatcatgatacggtgagtgaaagcccttgtgaagtgaaggtatatggatgagtgctgaatgatatcacaagcaaccgggacaaatgctcgaggtgagttgttaactttatcatgtgtctgaggatgagcaacgagctaacatacaacagaccatcgtcgagccctccttcaaacccctagcctacgccctaatcctcctccgccaaggaggccatccatgtgtcttctacggcgacctctacggcacctgcgacggcgaccacccgccaactcccgcctgcgagggccaacttccgaatctgatgcgtgcccgcaagctgtacgcctacggtgagcaggaggactacttcgaccagcccaactgtattggtaagtcaagccctatccagtctgcgacatagtctgataaatgagaaggattcatccgctacggcaacgccgcccacccctccggactggcctgcgtcatgagcaacggcggaccagccacgaagcgcatgtacgtgggtcggaaacatgccggcgagaagtggacggatctgctgcagcgaggcggtgatcacccgtctgtcacggtgattgatgaaatggggtacggggagtttccggttcagagtatgagggtgagtgtctgggtggatagtgcggcagatggccgagagggtgtcggggcagaattgtacgtctataccctattccacttattgctcttggtagtcgtgggggtggatgtttaaaactgatccaggggatgatgatgatgctaatattggaagtttagtgacgtcgatatctacggcattcaggcactataactaacccacccggtacttactcattattgagaaaataatggtgtccatatatatctctaagacacgatgtatattaatacaattcacaatgtaactatttcccagactagttttactatagtaagacaatacaacaatactacagagtaataatcaatgtatccacacaaactagtctactccatctattctacaactaactccaagacacacaatcacacacagtaagacacaatctcacccaaaaataatccctccattcatccaagcgagcgagaaacccaaaacccgaaagctaaaagaaaagaagaaaaagattctggaccctctttacccaatccagtaacccattcggtgctgcccgacactggta

SEQ ID NO: 5 - DNA, 3010 bp, Aspergillus niger
aaataaccattccatcctcaccatcaagtgacgaaacctgccatggccgccaagccgtgctgcccactgattggtgcagtttctcctttcggtcaacggtaagccgacggagaacttctgcttggttcataacgatttgtgcggtttcatcccctcaggcatcaacattttatcctcggaattggtgtctagcgttgcgacgcgattgatgcttgacctacatacttagaccctagaccagtatacgtggtggcatctgcaaggaacgctctagaaatcataggagaacctcctagggatatgttttgcgcccggaaggcacagcattcagccccatctactatttaaagtatcaacacactaaaacaggaggctttagtggctgtttttctgatcgaccatcataccttaaatcataccagacagctatcaccaatatgacgtgttttgcagtatatacatttgatacatctaagacctgaaccacggtggaggctctctttttcatcaccgaatcaatctttctcagcttcacactcgaaataggatacttctccttccttttcacccttgttgataccgtttctgaaagtagaagtacggcattttctccatgttatctttcctcctatggtgccatccaaagaagaggaaggagagacaactatggaagcagattgaaggttagcctccgtaatcatcaaggtgcattttattgcaagttgaatccccgcttacgtgtccaccaacagaagaagctgagcacttggaccagcttccatcatgggatgctcccgacaacactttaatgctgcaagcctttgaatggcatgtgccagctgaccaaggtcactggcgtcgtcttcaccaggccttaccaaacttcaaggcgattggggtagacaacatatggatcccgcccgggtgtaaagctatgaatccatctggtaacggatatgatatttacgatttgtatgacttgggagaattcgagcaaaaggggtctcgagctactaaatggggtaccaaagaggagctccagtccttggtagctgcagcccaagatttcggtataggtatttactgggacgctgtcctcaatcacaaggcgggagcagattacgcagagcgctttcaagctgtcagggttgacccacagggtatgccatggccttattcgactagtttcgggtgcacctgttaacttggaattgtctagagcgtaatatgaaaatcgcccctgcagaggaaattgaaggctgggtgggattcaacttctctgggcgtggcaaccactatagttcgatgaagtacaacaaaaaccacttcagcggtatcgactgggaccagtcgcgtcaaaaatgcggagtctacaagatccaaggacatgaatgggcgaacgacgtcgccaatgagaacggaaattacgactatctcatgttcgccaacttggactactccaacgcagaagtacgacgcgatgttctgaaatgggccgagtggctcaatgctcaattgcctctaagcggcatgaggttggatgcggtcaaacattactcggctggttttcagaaagagcttattgatcatcttcgaactattgctgggccagactatttcatagtgggcgagtactggaaaggcgagaccaagccgttagttgactacctgaagcagatggactacaagctatcattgttcgattccgctctggttgggcggttctcaagcatttcacagacaccaggggcggatcttcgcaacattttctataatacattggtccaattgtacccagatcattctgtcgtaagttgattgtcattttggtgagatcccttactgaccactaaaagactttcgttgcaaaccatgatactgtaggtgcaatatgccctttggcctcattctactaacatagatccaaagcaaccaggtcaatccctcgaagtacgcacaccgcccactttgattttacagatctaacagatgttcaggcgccagtaacatcattcttcaaacccctcgcgtacgcccttatccttctccgtgaccaagggcaaccatgcatattctacggagacctttacggcctccaagccgatgtcaaagatccaatgacaccgtcttgcaggggcaagctgtccatcctcacccgagctcgaaagctctacgcatatggcctgcaacgagattattttgacaagccgaactgcatcggttagtgcctgctttcgttatcctctaccgcacactgacaggagtaggttttgtccgctatggtaaccgtcggcatccctctggtcttgcatgcgtgatgagcaatgcgggtccgtcgaggaagcggatgtatgttggtcgacgacacgccaagcaaacatggaccgatatcctgcagtggtgtgatcagactgttgtcattgatgccaagggatatggagagtttccggttagtgcgatgagcgtgagtgtatgggtgaactccgaggctgaggggagagatagcctctcacatcatttgtatgtccctgctcacagtcttagtacggatgatttgctgacgagtgctgaaattagtgatgagaacatatacaaactagcttaatcgccttgggtatctgtgatgactactagacaatccccaaagaaacataatacacgatcatctatacaacaacctctctaaccaaccaatcaaaccaacaacggcgtatcaaagcacccaaaccccttgaccacactcatctcatccacatccgcaaacttcgtctttaacgaccagcccacagtttcatttctcaaaatctcccccttcttccaaatacacagcccaaacacccccacttccgaaatcacatccgattcctccaccccttcccgcgacatcagcacattccccacaacaggcgacctaatcctctccatcaacacatacctaccccattcggattccgaactcattcccaacagaaaccccgggatatcctccccgtagatattatgccccccg

SEQ ID NO: 6 - DNA, 3001 bp, Aspergillus niger
tctgttggttcggggtccagaagtaccaatacccaattattcccactttacccaatggtcgaataatacacaatacccattacacaattcccgatgccgatgtcacgtaagtttccatacggtccggatacatcggtatcatcttcgctgaagtgttgttcactggtcggtatcagccttcttgccctgaacagacacaaaggatgtatgtgtccgcttcgtatatgaacatgctcaacaggaggttacgcaggtcataaccatcaagacattttttctcacctctttccagctaataatcccaccattgaactactctccttgccttctttcatctgcgcatgcaaacgccaccaaccatggacgacgggtggtggatcatctcgttgctggtggtaactctgggaatccccacggttaatgcggcttccagagaccaatggatcggtcgctctatctatcaaatcgtgaccgatcgctttgctcgatcggataactcaaccaccgctgcttgtgatgcagcactaggaaactactgcggaggctctttccagggcatcatcaataagctggactacatccaggagctcgggtttgatgcggtaggtttaccaccatcatcctgcctctcgctctctgccactctttctcatacctgaaacaccagatatggatctctcccgcacaaagccaaatttccgcccgaacagcagatctctcaggtatggcactttcaaaatcttcacaaaccatctgcaactctgtacattgttatatacacgatatactatgtagcttggtaggtaagaaattcaacatgttaacggttcatagcataccatggatattggcccaatgatctgtattccatcaactctcattttggcactcccaaggagctggaagccttgtcctctgccctgcacgatcgtggcatggtgagtcgctctgccattttccctaaagatgacactgaccgcgcaaaacccagtacttgatgcttgacatcgtggttggtgatatggcctgggcgggaaatcacagcaccgtcgattacagcaactttaatcctttcaatgatcagaagtttttccatgatttcaagctcctctccagtgaccccacaaatgaaacttgtgttctggatgtaagtctgtattgtgaatcatgacccgtgggattttgctaaccccatatgaagtgctggatgggagacaccgtcgtatcacttcccgatctgcgaaatgaagaccaacaagttcagaatattcttggtacctggatttcggggttggtttcgaactactcaagtaagacttcttccttggccgttatggaatattgattgacctgtttctgttttgtagttgacggactgcgtattgacagcgtcttaaatatcgccccggacttcttctccaacttcaccaagtcatcaggggttttcactgtcggtgaaggtgccacggccgatgcggctgatgtttgccctctgcagccaagtttaaatgggcttttgaattatccattgtaagtaccgcatcgtggtgagtaaagtcagtcgtgttgactttctcaggtactatattcttaccgacgccttcaacacgaccaacgggaacctgagcaccattaccgagtccataagctacaccaaaggacagtgcgaggtagggcatgccttcgaattggattataagggcaaactaatcatatacaggatgtcttggctctggggacgtttactgcaaaccaggatgttccgcgcttcggttcatacacgtcggatatttcggtaagttcagtcggtggtcctatcttggtactgaacaatgtttgactttattgcttgtgagcagctggcgcgcaatatcctgaccagcagtatgctgacggacggcatccccatccgtatgttccgatccccgcctcccccatagtaagcaagattcctcagacaccactgataatcaaaatacagtctattacggcgaggagcagcatttgacaggatcctacaatcctgtcaaccgtgaggctctgtggctgaccaactactcgatgcgctcgacctccctccccaccctcgtccaatccctgaaccgccttcgatcgtatgctagcggggacggtgagcagtacacgcaaaagtcgcaatctgggagcgattacctctcgtacctgtcagcacccatttacaattctacgcacattctggccacgcgcaaggggtttgcgggcaatcagatcgtgagtgtcgtatccaatctgggagccaagccagccagcaaagccaccacgaaaatcacgttgggctcagacgagactggattccaatccaagcagaatgtgaccgagatcttatcgtgcaagacgtatgtcacggattccagcgggaatctggcagtggatctgagctcggacgggggtccgcgcgtgtactatcccacggacagcctgaaggacagcactgatatctgtggtgatcagaccaaatcggcgaccccgagtagctccgcagcctcgtccgcgagcttgacccagtccaagggttcagagacctgtttgtttggggtgccgttggggataagcacattggtggtcacagttgcgatggccacgtcctacgtgttctagtctccttgagcctctaccccttctgtttgtcgcctgtctttccctgtttcgcattacccctatgattattgtttgctcataacgctcttttgaatgttttgaaactgtggctttacttgaaacccgataccacccctctggctgcactgatacattactaagtatagaatgtggcgtcttgtctcttacgagtcaccctagcatcacggtaaaataaatgtgttgtttccgtctgagtctcttgaaatcttttgtagcacgggggaaagaaaattgtcagggtctccttctggcacggatggcatagacgaagcagtatacagggaggtaaattcgttcgggtttcccgactgttacgggcttt

SEQ ID NO: 7 - DNA, 1485 bp, Aspergillus niger
atgacaatctttctgtttctggccattttcgtggctacagctctggcagccacgcctgcagaatggcgctcccagtcgatatatttcctgctcaccgatcgctttgcgcgaacggataattctaccactgcttcttgtgacttgagcgctcggcaatattgcggtggatcctggcagggcatcatcaatcagctggactatattcaaggaatgggctttacagcgatctggatcacacccgtaactgcacagatcccccaagatactggttacggacaggcatatcacggatactggcagcaggacgcttatgccctgaactcccattatggtacggcagacgatctcaaagctctggcttcagctcttcactcacggggcatgtatctcatggtggacgttgttgccaatcacatgggccacaatggtacggggagctctgtggactacagtgtttataggccatttaattcgcaaaagtactttcacaacctctgttggatctctgattacaataaccagacaaacgttgaagactgctggctaggcgataacaccgttgccttgccggatcttgatactaccagtacggaggtgaagaatatgtggtatgactgggtcgagtctctcgtctctaactactccgtcgacggcctccgcgtagacacagtcaagaacgtacagaagaacttctggcccggctacaacaatgcttcaggcgtgtactgtattggagaagtcttcgatggggacgcctcatacacctgtccttatcaggaagacttggacggagtccttaattaccccatgtactatccactcctccgggctttcgaatccaccaacggcagtatcagcgacctctataacatgatcaacaccgtgaaatccacctgcagagattctacgcttctagggaccttcgtcgaaaaccacgataacccacgctttgccaaaagctacacaagcgacatgtccctagccaaaaatgccgcaacattcactatcctggctgacggcattcccatcatatacgccggtcaggaacagcactatagcggcggtaatgacccctacaaccgcgaagcgacctggctttcaggctacaagaccaccagcgagctctacacccatatcgccgcatcgaacaagattcgcacccacgctataaaacaggataccggatatctcacctacaaaaactaccccatctaccaagacacctcgacccttgccatgcgcaaaggctacaatggcacccaaactatcacggtcctttctaaccttggcgcctcggggtcctcatacacactctccctcccaggaacaggctacacagccggccaaaagattactgaaatctatacctgcacgaatctaacagtcaactcaaatggctcggtgccagtacccatgaagagcgggttaccgcggatcctctatcctgcagataagttggttaatggaagctcattttgcagttag

SEQ ID NO: 8 - DNA, 1650 bp, Aspergillus niger
atgtttcgaaaatccgcttccctcctgggccaacggctcatggccgtttgtctcctgtgctggtgcgtttcgctagcgaccgccgcaagcacagaagaatggaagacgcgatccatctaccagacgatgacggatcggtttgccctcaccaacggttcgacgaccgcaccatgcaatactacggtagccaactactgtggcgggtcttggcaggggacgatcgataagctggactacatccaaggcatggggttcgatgcgatcatgatctctcccgtaattaaaaacattgcggggcgatctaaggacggtgaggcctaccatggatactggcctctggatctatacgagatcaattctcatttcggaacccgggaggaactgttgaagctgagcgaggagatccacgcacgcggcatgtacttgctgctggacgtcgtcatcaataatatggcttacatgacggacggcgaggatcctgcgacgaccattgactacaatgttttccctcagttcaatggatcttcatacttccacccctactgtcttattacgaactggaataactatacggacgcgcagtggtgtcagactggtgacaattatactgcactcccagatctgtacacagaacacaccgcggtgcagaacatcttaatggactggagtaaatcggtcatcagcaattactccgtcgacgggctacgaatcgacgctgccaagtccctcactcccagctttctgcctacatatgcgagtaccgtgggcggattcatgaccggcgaagtcatggattcgaacgccaccaacgtgtgcaaatatcagacggattacctgccaagtcttccaaactaccccctctattactccatgatcacggccttcctcaacggcgagcctgcgaccttgctcgaggaaatcgcgacgatcaatgatctttgccctgatacgttcgcaatggtcaacttcatcgaagatcaagacgttgaccgatgggcctacatgaatgacgacatcatgctagctaaaactgcactgaccttcatgatgctctacgacggtattcccttggtctaccagggtctggagcaggccattgcctattccaaccgagcggccttgtggttgacagatttcgacaccaatgcgacgctttataaacacatcaagaaactcaatgccatccgcaaacatgccattaaccttgattccagctacatcagttcgaaaacatatcccatctatcaaggaggtagcgagttggctttctggaagggcaacaatggacgccaagtcatcatggttttatctacggcgggctcgaatggttcagcttatactctgacactacccgtaagctatggggctagtgaggtggtcacggaagtgctgaactgcgtcaattatacggtcaacacttacagtcaattggtcgtggacatggataagggcgaacctcgggtcttctttcccgcttcgatgatgccggggagcgggctgtgtggctacaatacttctaatgtgacgtattctgagctgagacttgctgctgtgggatcttcgtcgtctgctggtagccactctgtcataccgtctgcttttgcttcgcttttcatggcgatagttgcatttttggcattccggatataa

SEQ ID NO: 9 - DNA, 1668 bp, Aspergillus niger
atggtctcaatgtcggccctgcggcacgggcttggggtcctctatcttgctagctggctggggtcgtcgctggctgccagcaccgagcaatggaagtcccggtcaatctatcagaccatgacggatcggttcgcgcgcactgatggctcaaccacctccccctgcaacaccacggagggtctgtactgtggtggtacctggcgcggcatgatcaaccatctggattacatccaggggatgggctttgatgccgtcatgatctcccctatcatcgagaacgtcgaaggtcgcgtggagtacggagaagcctatcacggctattggcccgtggatctgtactccctcaactcgcactttggaacccaccaggatctactggacctgagtgatgccctgcatgctcgcgacatgtatctgatgatggacacagtcatcaacaacatggcctacatcaccaatggctccgaccccgccacccacatcgactactcaaccctgacccccttcaatagctcgtcttactaccatccctactgtaagatcaccgactggaataacttcaccaatgcccagctgtgccaaaccggtgacaacatcgtggccttgcccgatctgtataccgagcatgccgaggtgcaagaaaccctgagcaactgggccaaggaagtcatctccacctattccatcgacggcctccgcatcgatgccgcaaaacatgtgaaccccggcttcctgaagaatttcggcgatgcgcttgatatcttcatgaccggcgaagtgctgcagcaagaggtcagcacaatctgcgattatcagaacaactacatcggcagtctcccgaattaccccgtctactacgccatgttgaaggcctttaccttgggcaacaccagcgccctggccactcaggtccagtcgatgaagaattcctgtaatgatgtgaccgccttgtcgtcattctccgaaaaccacgatgtcgcacgatttgccagcatgacccatgacatggctctcgcgaagaatattctcacattcactcttctcttcgacggtgttcccatgatctatcaaggtcaagagcagcatctcgacggccctggcagtccggaaaaccgagaagcgatatggctctctgagtacaacaccgacgccgagctctacaagttgatcggcaagttgaacgccatccggaagcacgcctaccgactcgacaatcactaccccgatgtcgagacgtaccccatcttcgaaggtggcagtgaactgggattccgcaagggaatcgaaggtcgacaggtggtcatgcttctgtccacgcaaggcacgaacagcagcgcttacaacctctcgatgccggtgagtttcacgggtggcacagtagtgaccgagattctgaactgcgtcaactacacggtcaacacccagagcgaactcgtggttccgatggataaaggagagccgcgcgtgttcttcccggcggatctgatgcccggcagtgggctgtgcggacttcctgtcgcgaatgtcacctatgctgccttgaggacgcagggtgcagcggcggcggaggctgccttgtcgctaggtatcaagactgatgcagcttctagtgctttgctttctctggggctgtctgtggtggcgggtctgattgtggggatgtggtaa

SEQ ID NO: 10 - DNA, 1629 bp, Aspergillus niger
atgttcagtttcctcccctgcttcaaaactcgccgacaacgcaccaaatcccaaactcaatcccaaaagcaaatagaggaaaaagccaacgccatagaatccctcccctcatggcactcccccacggaaaacaccctcctatttcaagccttcgaatggcacgtccccgcaacccccaacaccgcggacaagcgcagccactggcgccggctccagcatgccttaccagctatccactccctcggtgtaaccagcatctggataccgcctggatgcaagggcatggacacaaatggaaatgggtatgatatctacgatttatatgatctaggcgaattcgaccagaaaggagcagtgcgaacgaaatggggcacgcgcggagaattagaggatcttgttcgagatgcaaatgctctgggcgtaggtgtattatgggatgcggtgttgaatcataaggctggggcggatagcgtggagaggttcgagggacgagatatcgaagatggtaatccccaacaaatctccggctggacgtccttcaccttccccggccgcggcaccacatacagcccccttcaataccactggcagcacttcagcggcgtcgactgggacgacgcccaacaacgcaaagcaatctacaagatcctcgacccttcccgccccgacaagaactgggcccaggacgtcggcacagacgagaacggcaactatgactacctgatgttcgcggacctggacttctcacacccggaggtccgggaggatgtactgcgctgggggaagtggattatgtctgtgttgccgttgagcggaatgcggttagatgcggcgaagcatttctcgacggcatttcagagggactttattgattgtgtgcggcaggaggccggggacaggaaggtatctggaggagatggagtatcgggtggcggcggtggatgtgccgctggtagagaggttctcgaggttgtcgagggtgaagagggcggatttgaggggagtgttgagggggacgttggtggagagcaggccggggaatgcgttggtgagtttatcccagtgatccaattcgggggtggtgaggctgatcaaccgggacaaatgctcgagaccatcgtcgagccctccttcaaacccctagcctacgccctaatcctcctccgccaaggaggccatccatgtgtcttctacggcgacctctacggcacctgcgacggcgaccacccgccaactcccgcctgcgagggccaacttccgaatctgatgcgtgcccgcaagctgtacgcctacggtgagcaggaggactacttcgaccagcccaactgtattggattcatccgctacggcaacgccgcccacccctccggactggcctgcgtcatgagcaacggcggaccagccacgaagcgcatgtacgtgggtcggaaacatgccggcgagaagtggacggatctgctgcagcgaggcggtgatcacccgtctgtcacggtgattgatgaaatggggtacggggagtttccggttcagagtatgagggtgagtgtctgggtggatagtgcggcagatggccgagagggtgtcggggcagaatttgacgtcgatatctacggcattcaggcactataa

SEQ ID NO: 11 - DNA, 1695 bp, Aspergillus niger
atgttatctttcctcctatggtgccatccaaagaagaggaaggagagacaactatggaagcagattgaagaagaagctgagcacttggaccagcttccatcatgggatgctcccgacaacactttaatgctgcaagcctttgaatggcatgtgccagctgaccaaggtcactggcgtcgtcttcaccaggccttaccaaacttcaaggcgattggggtagacaacatatggatcccgcccgggtgtaaagctatgaatccatctggtaacggatatgatatttacgatttgtatgacttgggagaattcgagcaaaaggggtctcgagctactaaatggggtaccaaagaggagctccagtccttggtagctgcagcccaagatttcggtataggtatttactgggacgctgtcctcaatcacaaggcgggagcagattacgcagagcgctttcaagctgtcagggttgacccacaggagcgtaatatgaaaatcgcccctgcagaggaaattgaaggctgggtgggattcaacttctctgggcgtggcaaccactatagttcgatgaagtacaacaaaaaccacttcagcggtatcgactgggaccagtcgcgtcaaaaatgcggagtctacaagatccaaggacatgaatgggcgaacgacgtcgccaatgagaacggaaattacgactatctcatgttcgccaacttggactactccaacgcagaagtacgacgcgatgttctgaaatgggccgagtggctcaatgctcaattgcctctaagcggcatgaggttggatgcggtcaaacattactcggctggttttcagaaagagcttattgatcatcttcgaactattgctgggccagactatttcatagtgggcgagtactggaaaggcgagaccaagccgttagttgactacctgaagcagatggactacaagctatcattgttcgattccgctctggttgggcggttctcaagcatttcacagacaccaggggcggatcttcgcaacattttctataatacattggtccaattgtacccagatcattctgtcgtgcaatatgccctttggcctcattctactaacatagatccaaagcaaccaggtcaatccctcgaagcgccagtaacatcattcttcaaacccctcgcgtacgcccttatccttctccgtgaccaagggcaaccatgcatattctacggagacctttacggcctccaagccgatgtcaaagatccaatgacaccgtcttgcaggggcaagctgtccatcctcacccgagctcgaaagctctacgcatatggcctgcaacgagattattttgacaagccgaactgcatcggttttgtccgctatggtaaccgtcggcatccctctggtcttgcatgcgtgatgagcaatgcgggtccgtcgaggaagcggatgtatgttggtcgacgacacgccaagcaaacatggaccgatatcctgcagtggtgtgatcagactgttgtcattgatgccaagggatatggagagtttccggttagtgcgatgagcgtgagtgtatgggtgaactccgaggctgaggggagagatagcctctcacatcatttgtatgtccctgctcacagtcttagtacggatgatttgctgacgagtgctgaaattagtgatgagaacatatacaaactagcttaa

SEQ ID NO: 12 - DNA, 1704 bp, Aspergillus niger
atggacgacgggtggtggatcatctcgttgctggtggtaactctgggaatccccacggttaatgcggcttccagagaccaatggatcggtcgctctatctatcaaatcgtgaccgatcgctttgctcgatcggataactcaaccaccgctgcttgtgatgcagcactaggaaactactgcggaggctctttccagggcatcatcaataagctggactacatccaggagctcgggtttgatgcgatatggatctctcccgcacaaagccaaatttccgcccgaacagcagatctctcagcataccatggatattggcccaatgatctgtattccatcaactctcattttggcactcccaaggagctggaagccttgtcctctgccctgcacgatcgtggcatgtacttgatgcttgacatcgtggttggtgatatggcctgggcgggaaatcacagcaccgtcgattacagcaactttaatcctttcaatgatcagaagtttttccatgatttcaagctcctctccagtgaccccacaaatgaaacttgtgttctggattgctggatgggagacaccgtcgtatcacttcccgatctgcgaaatgaagaccaacaagttcagaatattcttggtacctggatttcggggttggtttcgaactactcaattgacggactgcgtattgacagcgtcttaaatatcgccccggacttcttctccaacttcaccaagtcatcaggggttttcactgtcggtgaaggtgccacggccgatgcggctgatgtttgccctctgcagccaagtttaaatgggcttttgaattatccattgtactatattcttaccgacgccttcaacacgaccaacgggaacctgagcaccattaccgagtccataagctacaccaaaggacagtgcgaggatgtcttggctctggggacgtttactgcaaaccaggatgttccgcgcttcggttcatacacgtcggatatttcgctggcgcgcaatatcctgaccagcagtatgctgacggacggcatccccatcctctattacggcgaggagcagcatttgacaggatcctacaatcctgtcaaccgtgaggctctgtggctgaccaactactcgatgcgctcgacctccctccccaccctcgtccaatccctgaaccgccttcgatcgtatgctagcggggacggtgagcagtacacgcaaaagtcgcaatctgggagcgattacctctcgtacctgtcagcacccatttacaattctacgcacattctggccacgcgcaaggggtttgcgggcaatcagatcgtgagtgtcgtatccaatctgggagccaagccagccagcaaagccaccacgaaaatcacgttgggctcagacgagactggattccaatccaagcagaatgtgaccgagatcttatcgtgcaagacgtatgtcacggattccagcgggaatctggcagtggatctgagctcggacgggggtccgcgcgtgtactatcccacggacagcctgaaggacagcactgatatctgtggtgatcagaccaaatcggcgaccccgagtagctccgcagcctcgtccgcgagcttgacccagtccaagggttcagagacctgtttgtttggggtgccgttggggataagcacattggtggtcacagttgcgatggccacgtcctacgtgttctag

SEQ ID NO: 13 - protein, 494 aa, Aspergillus niger
MTIFLFLAIFVATALAATPAEWRSQSIYFLLTDRFARTDNSTTASCDLSARQYCGGSWQGIINQLDYIQGMGFTAIWITPVTAQIPQDTGYGQAYHGYWQQDAYALNSHYGTADDLKALASALHSRGMYLMVDVVANHMGHNGTGSSVDYSVYRPFNSQKYFHNLCWISDYNNQTNVEDCWLGDNTVALPDLDTTSTEVKNMWYDWVESLVSNYSVDGLRVDTVKNVQKNFWPGYNNASGVYCIGEVFDGDASYTCPYQEDLDGVLNYPMYYPLLRAFESTNGSISDLYNMINTVKSTCRDSTLLGTFVENHDNPRFAKSYTSDMSLAKNAATFTILADGIPIIYAGQEQHYSGGNDPYNREATWLSGYKTTSELYTHIAASNKIRTHAIKQDTGYLTYKNYPIYQDTSTLAMRKGYNGTQTITVLSNLGASGSSYTLSLPGTGYTAGQKITEIYTCTNLTVNSNGSVPVPMKSGLPRILYPADKLVNGSSFCS

SEQ ID NO: 14 - protein, 549 aa, Aspergillus niger
MFRKSASLLGQRLMAVCLLCWCVSLATAASTEEWKTRSIYQTMTDRFALTNGSTTAPCNTTVANYCGGSWQGTIDKLDYIQGMGFDAIMISPVIKNIAGRSKDGEAYHGYWPLDLYEINSHFGTREELLKLSEEIHARGMYLLLDVVINNMAYMTDGEDPATTIDYNVFPQFNGSSYFHPYCLITNWNNYTDAQWCQTGDNYTALPDLYTEHTAVQNILMDWSKSVISNYSVDGLRIDAAKSLTPSFLPTYASTVGGFMTGEVMDSNATNVCKYQTDYLPSLPNYPLYYSMITAFLNGEPATLLEEIATINDLCPDTFAMVNFIEDQDVDRWAYMNDDIMLAKTALTFMMLYDGIPLVYQGLEQAIAYSNRAALWLTDFDTNATLYKHIKKLNAIRKHAINLDSSYISSKTYPIYQGGSELAFWKGNNGRQVIMVLSTAGSNGSAYTLTLPVSYGASEVVTEVLNCVNYTVNTYSQLVVDMDKGEPRVFFPASMMPGSGLCGYNTSNVTYSELRLAAVGSSSSAGSHSVIPSAFASLFMAIVAFLAFRI

SEQ ID NO: 15 - protein, 555 aa, Aspergillus niger
MVSMSALRHGLGVLYLASWLGSSLAASTEQWKSRSIYQTMTDRFARTDGSTTSPCNTTEGLYCGGTWRGMINHLDYIQGMGFDAVMISPIIENVEGRVEYGEAYHGYWPVDLYSLNSHFGTHQDLLDLSDALHARDMYLMMDTVINNMAYITNGSDPATHIDYSTLTPFNSSSYYHPYCKITDWNNFTNAQLCQTGDNIVALPDLYTEHAEVQETLSNWAKEVISTYSIDGLRIDAAKHVNPGFLKNFGDALDIFMTGEVLQQEVSTICDYQNNYIGSLPNYPVYYAMLKAFTLGNTSALATQVQSMKNSCNDVTALSSFSENHDVARFASMTHDMALAKNILTFTLLFDGVPMIYQGQEQHLDGPGSPENREAIWLSEYNTDAELYKLIGKLNAIRKHAYRLDNHYPDVETYPIFEGGSELGFRKGIEGRQVVMLLSTQGTNSSAYNLSMPVSFTGGTVVTEILNCVNYTVNTQSELVVPMDKGEPRVFFPADLMPGSGLCGLPVANVTYAALRTQGAAAAEAALSLGIKTDAASSALLSLGLSVVAGLIVGMW

SEQ ID NO: 16 - protein, 542 aa, Aspergillus niger
MFSFLPCFKTRRQRTKSQTQSQKQIEEKANAIESLPSWHSPTENTLLFQAFEWHVPATPNTADKRSHWRRLQHALPAIHSLGVTSIWIPPGCKGMDTNGNGYDIYDLYDLGEFDQKGAVRTKWGTRGELEDLVRDANALGVGVLWDAVLNHKAGADSVERFEGRDIEDGNPQQISGWTSFTFPGRGTTYSPLQYHWQHFSGVDWDDAQQRKAIYKILDPSRPDKNWAQDVGTDENGNYDYLMFADLDFSHPEVREDVLRWGKWIMSVLPLSGMRLDAAKHFSTAFQRDFIDCVRQEAGDRKVSGGDGVSGGGGGCAAGREVLEVVEGEEGGFEGSVEGDVGGEQAGECVGEFIPVIQFGGGEADQPGQMLETIVEPSFKPLAYALILLRQGGHPCVFYGDLYGTCDGDHPPTPACEGQLPNLMRARKLYAYGEQEDYFDQPNCIGFIRYGNAAHPSGLACVMSNGGPATKRMYVGRKHAGEKWTDLLQRGGDHPSVTVIDEMGYGEFPVQSMRVSVWVDSAADGREGVGAEFDVDIYGIQAL

SEQ ID NO: 17 - protein, 564 aa, Aspergillus niger
MLSFLLWCHPKKRKERQLWKQIEEEAEHLDQLPSWDAPDNTLMLQAFEWHVPADQGHWRRLHQALPNFKAIGVDNIWIPPGCKAMNPSGNGYDIYDLYDLGEFEQKGSRATKWGTKEELQSLVAAAQDFGIGIYWDAVLNHKAGADYAERFQAVRVDPQERNMKIAPAEEIEGWVGFNFSGRGNHYSSMKYNKNHFSGIDWDQSRQKCGVYKIQGHEWANDVANENGNYDYLMFANLDYSNAEVRRDVLKWAEWLNAQLPLSGMRLDAVKHYSAGFQKELIDHLRTIAGPDYFIVGEYWKGETKPLVDYLKQMDYKLSLFDSALVGRFSSISQTPGADLRNIFYNTLVQLYPDHSVVQYALWPHSTNIDPKQPGQSLEAPVTSFFKPLAYALILLRDQGQPCIFYGDLYGLQADVKDPMTPSCRGKLSILTRARKLYAYGLQRDYFDKPNCIGFVRYGNRRHPSGLACVMSNAGPSRKRMYVGRRHAKQTWTDILQWCDQTVVIDAKGYGEFPVSAMSVSVWVNSEAEGRDSLSHHLYVPAHSLSTDDLLTSAEISDENIYKLA

SEQ ID NO: 18 - protein, 567 aa, Aspergillus niger
MDDGWWIISLLVVTLGIPTVNAASRDQWIGRSIYQIVTDRFARSDNSTTAACDAALGNYCGGSFQGIINKLDYIQELGFDAIWISPAQSQISARTADLSAYHGYWPNDLYSINSHFGTPKELEALSSALHDRGMYLMLDIVVGDMAWAGNHSTVDYSNFNPFNDQKFFHDFKLLSSDPTNETCVLDCWMGDTVVSLPDLRNEDQQVQNILGTWISGLVSNYSIDGLRIDSVLNIAPDFFSNFTKSSGVFTVGEGATADAADVCPLQPSLNGLLNYPLYYILTDAFNTTNGNLSTITESISYTKGQCEDVLALGTFTANQDVPRFGSYTSDISLARNILTSSMLTDGIPILYYGEEQHLTGSYNPVNREALWLTNYSMRSTSLPTLVQSLNRLRSYASGDGEQYTQKSQSGSDYLSYLSAPIYNSTHILATRKGFAGNQIVSVVSNLGAKPASKATTKITLGSDETGFQSKQNVTEILSCKTYVTDSSGNLAVDLSSDGGPRVYYPTDSLKDSTDICGDQTKSATPSSSAASSASLTQSKGSETCLFGVPLGISTLVVTVAMATSYVF

(19) United States
(12) Patent Application Publication
(10) Pub. No.: US 2005/0032059 A1
(43) Pub. Date: Feb. 10, 2005

(54) Novel amylases and uses thereof

(76) Inventors: Dieter Maier, Martinsried (DE); Alexander Stock, Martinsried (DE); Christian Wagner, Martinsried (DE); Ulrike Folkers, Munchen (DE); Kaj Albermann, Martinsried (DE)

Correspondence Address: MORRISON & FOERSTER LLP, 3811 VALLEY CENTRE DRIVE, SUITE 500, SAN DIEGO, CA, 92130-2332, US

(21) Appl. No.: 10/486,868

(22) PCT Filed: Aug. 2, 2002
(86) PCT No.: PCT/NL02/00522

(30) Foreign Application Priority Data
Aug. 16, 2001 (EP) 01000379.6
Aug. 16, 2001 (EP) 01000380.4
Aug. 16, 2001 (EP) 01000381.2
Aug. 16, 2001 (EP) 01000382.0
Aug. 16, 2001 (EP) 01000383.8
Aug. 16, 2001 (EP) 01000384.6

(51) Int. Cl. C12Q 1/68; C07H 21/04; C12N 1/16; C12N 15/74

(52) U.S. Cl. 435/6; 435/69.1; 435/254.1; 435/320.1; 530/350; 536/23.7

(57) ABSTRACT

The invention relates to newly identified polynucleotide sequences comprising genes that encode novel amylases isolated from Aspergillus niger. The invention features the full length nucleotide sequences of the novel genes, the cDNA sequences comprising the full length coding sequence of the novel amylases as well as the amino acid sequence of the full-length functional proteins and functional equivalents thereof. The invention also relates to methods of using these enzymes in industrial processes and methods of diagnosing fungal infections. Also included in the invention are cells transformed with a polynucleotide according to the invention and cells wherein an amylase according to the invention is genetically modified to enhance or reduce its activity and/or level of expression.

### FIELD OF THE INVENTION

The invention relates to newly identified polynucleotide sequences comprising genes that encode novel amylases isolated from Aspergillus niger. The invention features the full length nucleotide sequence of the novel genes, the cDNA sequence comprising the full length coding sequences of the novel amylases as well as the amino acid sequences of the full-length functional proteins and functional equivalents thereof. The invention also relates to methods of using these enzymes in industrial processes and methods of diagnosing fungal infections. Also included in the invention are cells transformed with a polynucleotide according to the invention and cells wherein an amylase according to the invention is genetically modified to enhance its activity and/or level of expression.

### BACKGROUND OF THE INVENTION

Industrial processes for the hydrolysis of starch to glucose rely on inorganic acids or enzyme catalysis. The use of enzymes is preferred currently and offers a number of advantages associated with improved yields and favourable economics. Enzymatic hydrolysis allows greater control over amylolysis, the specifity of the reaction, and the stability of the generated products. The milder reaction conditions involve lower temperatures and near-neutral pH, thus reducing unwanted side reactions. Fewer off-flavor and off-color compounds are produced, especially 5-hydroxy-2-methylfurfuraldehyde, anhydroglucose compounds, and undesirable salts. Enzymatic methods are favored because they also lower energy requirements and eliminate neutralization steps.

Alpha-amylases (E.C. 3.2.1.1) or α-amylases catalyse the endohydrolysis of 1,4-alpha-glucosidic linkages in oligosaccharides and polysaccharides. They are also known as 1,4-alpha-D-glucan glucanohydrolase, Taka-amylase, endoamylase or glycogenase. Alpha amylases act on starch, glycogen and related polysaccharides and oligosaccharides in a random manner; reducing groups are liberated in the alpha-configuration.

Beta-amylases (E.C.3.2.1.2) catalyse the hydrolysis of 1,4-alpha-glucosidic linkages in polysaccharides so as to remove successive maltose units from the non-reducing ends of the chains. Other names are: 1,4-alpha-D-glucan maltohydrolase, Saccharogen amylase, Glycogenase. Beta amylases act on starch, glycogen and related polysaccharides and oligosaccharides producing beta-maltose by an inversion.

Glucoamylases (E.C.3.2.1.3) catalyse the hydrolysis of terminal 1,4-linked alpha-D-glucose residues successively from non-reducing ends of the chains with release of beta-D-glucose. Other names are: Glucan 1,4-alpha-glucosidase. 1,4-alpha-D-glucan glucohydrolase. Amyloglucosidase. Gamma-amylase. Lysosomal alphaglucosidase. Exo-1,4-alpha-glucosidase. Most forms of the enzyme can rapidly hydrolyse 1,6-alpha-D-glucosidic bonds when the next bond in sequence is 1,4, and some preparations of this enzyme hydrolyse 1,6- and 1,3-alpha-D-glucosidic bonds in other polysaccharides.

Amylases may conviently be produced in microorganisms. Microbial amylases are available from a variety of sources; Bacillus spec. are a common source of bacterial enzymes, whereas fungal enzymes are commonly produced in Aspergillus spec.

The low pH optimum of most fungal amylases permits the convenient use of acid conditions for the saccharification. Such conditions reduce unwanted isomerization reactions to fructose and other sugars that may reduce the glucose yield. Moreover, acid conditions restrict the growth of contaminating microorganisms in the saccharification reactors.

Amylases may be used in a manifold of industrial applications, including baking, brewing, the production of corn syrup and alcohol as well as in vinegar fermentation.

Malted wheat, barley, bacteria, and fungi are typical sources of α-amylase for baking purposes. Fungal α-amylase is added to bread doughs in the form of diluted powders, prepacked doses, or water dispersible tablets. The enzyme may be added to flours at the bakery or, more rarely, at the mill itself. Malted wheat and barley also can serve as sources of amylolytic activity when flours from these grains are blended with the final product at the mill. The properties of bacterial α-amylase permit its application to the production of coffee cake, fruit cake, brownies, cookies, snacks, and crackers. Fungal α-amylase, usually from A. oryzae, A. niger, A. awamori, or species of Rhizopus, is used to supplement the amylolytic activity in flour. Enzymes from these sources can raise the levels of fermentable monosaccharides and disaccharides of dough from a native level of 0.5% to concentrations that promote yeast growth. The sustained release of glucose and maltose by added fungal and endogenous enzymes provides the nutrients essential for yeast metabolism and gas production during panary fermentation. The A. oryzae α-amylase is sometimes favored for baking applications over the bacterial enzyme obtained from Bacillus species since the fungal enzyme is heat labile at 60-70° C. and does not survive the baking process. Its thermolability prevents enzymatic action on the gelatinised starch in the finished loaf which would cause a soft or sticky crumb. Bacterial α-amylase is also used with good results, but its dose must be measured carefully to avoid a bread with a gummy mouthfeel. Amylase supplementation is also beneficial and sometimes essential, since white bread flours contain 6.7-10.5% damaged starch. The added enzyme degrades damaged, ruptured starch granules that usually are present in bread flour more efficiently than does wheat β-amylase (Bigelis R. in: Enzymes in Food processing, Nagodawithana and Reed Eds. Acad. Press Inc p121-158 and references cited therein).

Amylase supplementation can improve other characteristics of bread quality, in addition to improving the quality of rolls, buns, and crackers, when used during manufacturing processes for these baked goods. In bread baking, treatment with fungal or bacterial amylase lowers the viscosity of bread dough, thereby improving the ease of manipulation by manual workers or machines. Measured doses of enzyme also lower the compressibility of the loaf, producing a softer bread. Further, such processing increases the bread volume by reducing the viscosity of the gelling starch and allowing greater expansion during baking before protein denaturation and enzyme inactivation fix the volume of the loaf. Favorable effects on taste, crust properties, and toasting qualities are observed. The storage characteristics of breads are changed also, yielding a product with a softer, more compressible crumb that firms more slowly and keeps longer, as determined by taste panels. Amylolytic activity also may elevate the sugar concentration in bread and yield a preferred sweeter product with sensory advantages (Bigelis R. in: Enzymes in Food processing, Nagodawithana and Reed Eds. Acad. Press Inc p121-158 and references cited therein).

In brewing, added enzymes contribute to the action of endogenous barley β-amylase and aid in the starch digestion process. Such added enzymes are especially important when nonmalted cereal grains such as corn and rice, termed adjuncts, are used. Since these adjunct grains are deficient in carbohydrases, fungal α-amylase and glucoamylase can increase starch digestion, reduce the proportion of unmalted grain, and insure a consistent quality of the mash. Amylase solubilizes barley amylose and amylopectin, exposing these substrates to further degradation by barley β-amylase. As a result, the levels of maltose and small dextrins are raised, eventually yielding the wort ingredients that promote yeast fermentation. Amylase preparations with low transglucosidase activity are favored since trace levels of this enzyme generate isomaltose and panose, both of which are nonfermentable by yeast. The source of amylase activity for brewing applications is generally enzyme from Aspergillus species such as A. niger or A. oryzae. Protease from these sources may be added in concert with amylase to solubilize protein and release amino acids essential for yeast proliferation. (Bigelis R. in: Enzymes in Food processing, Nagodawithana and Reed Eds. Acad. Press Inc p121-158 and references cited therein)

In the above processes, it is advantageous to use enzymes that are obtained by recombinant DNA techniques. Such recombinant enzymes have a number of advantages over their traditionally purified counterparts. Recombinant enzymes may be produced at a low cost price, high yield, free from contaminating agents like bacteria or viruses but also free from bacterial toxins or contaminating other enzyme activities.

Molecular cloning of amylases in fungi has been described. The DNA and deduced amino acid sequences of certain alpha-amylases from Aspergillus oryzae, A. niger and A. shirousamil are given in Wirsel et al. Mol. Microbiol 1989, (1) 3-14, Boel et al., Biochemistry 1990 (29) 6244-6249, and Shibuya et al., Biosci. Biotech. Biochem. 1992 (56) 174179. Molecular cloning of an α-amylase from Bacillus amyloliquefaciens is described by Takkinen et al. J. Biol. Chem. 1983, (258) 1007-1013.

It is important that amylases, in particular α-amylases, can be produced at low costs. This may be achieved by improving the production efficiency (higher expression levels) or by providing enzymes with an improved specific activity (higher activity per mg of enzyme). It is therefore an object of the present invention to provide improved enzymes with an improved production efficiency and/or improved specific activity.

When α amylases are used as bread improvers, it is advantageous to provide them, preferably together with other enzymes, in a liquid preparation. Enzyme stabilisers like glycerol are a major cost factor of liquid bread improvers and consequently there is a need for more stable α-amylases for use in such preparations in order to lower the amount of stabilisers. It is also an object of the present invention to provide more stable α-amylases.

α-Amylases are often used in combination with ascorbic acid, which tends to become unstable at higher pH values. α-Amylases on the other hand become unstable at lower pH values. As a compromise between the two requirements, such preparations are usually kept at a pH value around 4.7. It would therefore be advantageous to have α-amylases with a lower pH optimum and/or a higher stability at low pH values, preferably below pH 4.7. The present invention provides such enzymes.

Ascorbic acid is used in combination with α-amylases in many applications where it is converted into a number of chelating agents, e.g. oxalate. Oxalate is able to bind Ca ions and since α-amylases require Ca Ions for their stability, oxalate acts as a destabiliser for these α-amylase enzyme preparations. It is therefore an object underlying the present invention to provide enzymes that are less dependent on Ca ions for their stability.

Another characteristic of α-amylases according to the prior art is their limited thermostability. Fungal α-amylases are inactivated at about 65° C., therefore they are heat-inactivated at the beginning of the baking process. Also, this property makes fungal α-amylases unsuited for activity measurements in the Hagberg falling number method (AACC, 1983, Method 56-81 A) and the Brabender amylograph method (AACC, 1983, Method 22-1). Also, prolonged storage at temperatures slightly above room temperature sometimes deteriorates enzyme activity. It is therefore an object of the present invention to provide α-amylases with improved thermostability.

### OBJECT OF THE INVENTION

It is an object of the invention to provide novel polynucleotides encoding improved novel amylases. A further object is to provide improved naturally and recombinantly produced amylases as well as recombinant strains producing these. Also fusion polypeptides are part of the invention as well as methods of making and using the polynucleotides and polypeptides according to the invention.

### SUMMARY OF THE INVENTION

The invention relates to isolated polypeptides having α-amylase activity and one or more characteristics selected from the group consisting of:
1) An isolated polypeptide having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 or functional equivalents thereof,
2) An isolated polypeptide obtainable by expressing a polynucleotide having a nucleotide sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12 or a vector comprising said polynucleotides or functional equivalents thereof in an appropriate host cell, e.g. Aspergillus niger.
3) Polypeptide comprising a functional domain of a polypeptide according to (1) or (2)
4) An allelic variant of (1), (2) or (3),
5) A fragment of (1), (2), (3) or (4)
6) A polypeptide having improved specific activity and/or improved production efficiency expressed as enzyme activity per mg of purified enzyme or as enzyme activity per ml culture volume or per mg of biomass produced
7) A polypeptide with improved stability, preferably stable in the presence of less than 50% glycerol, preferably less than 40% glycerol, more preferably less than 30% glycerol, more preferably less than 20% glycerol, more preferably less than 10% glycerol, most preferably in the absence of glycerol
8) A polypeptide stable at pH values below 4.7, preferably below pH 4.0, even more preferably below pH 3.5,
9) A polypeptide with improved stability towards Ca ions.

It is expressly mentioned that α-amylases according to the invention may have one or more of the above characteristics. Methods for determining specific activity, production efficiency, stability, pH optimum and acid stability are well known in the art. Among others they may be found in the materials and methods section of WO 00/60058.

The invention also relates to polynucleotides encoding any of the polypeptides mentioned above.

More in particular, the invention provides for polynucleotides having a nucleotide sequence that hybridises preferably under highly stringent conditions to a sequence having a nucleotide sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12. Consequently, the invention provides nucleic acids that are about 40%, preferably 65%, more preferably 70%, even more preferably 75%, 80%, 85%, 90%, 95%, 96%, 97%, 98% or 99% homologous to any sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12.

In a more preferred embodiment the invention provides for such an isolated polynucleotide obtainable from a filamentous fungus, in particular A. niger is preferred.

In one embodiment, the invention provides for an isolated polynucleotide comprising a nucleic acid sequence encoding a polypeptide with having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 or functional equivalents thereof.

In a further preferred embodiment, the invention provides an isolated polynucleotide encoding at least one functional domain of a polypeptide having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 or functional equivalents thereof.

In a preferred embodiment the invention provides an amylase gene having a nucleotide sequence selected from the group consisting of SEQ ID NO: 1, SEQ ID NO: 2, SEQ ID NO: 3, SEQ ID NO: 4, SEQ ID NO: 5 or SEQ ID NO: 6. In another aspect the invention provides a polynucleotide, preferably a cDNA encoding an A. niger amylase having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 or variants or fragments of that polypeptide. In a preferred embodiment the cDNA has a nucleotide sequence selected from the group consisting of SEQ ID NO: 7, SEQ ID NO: 8, SEQ ID NO: 9, SEQ ID NO: 10, SEQ ID NO: 11 or SEQ ID NO: 12 or functional equivalents thereof.

In an even further preferred embodiment, the invention provides for a polynucleotide comprising the coding sequence of the polynucleotides according to the invention, preferred is a polynucleotide sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12.

The invention also relates to vectors comprising a polynucleotide sequence according to the invention and primers, probes and fragments that may be used to amplify or detect the DNA according to the invention.

In a further preferred embodiment, a vector is provided wherein the polynucleotide sequence according to the invention is functionally linked with regulatory sequences suitable for expression of the encoded amino acid sequence in a suitable host cell, such as A. niger or A. oryzea. The invention also provides methods for preparing polynucleotides and vectors according to the invention.

The invention also relates to recombinantly produced host cells that contain heterologous or homologous polynucleotides according to the invention.

In another embodiment, the invention provides recombinant host cells wherein the expression of an amylase according to the invention is significantly increased or wherein the activity of the amylase is increased.

In another embodiment the invention provides for a recombinantly produced host cell that contains heterologous or homologous DNA according to the invention and wherein the cell is capable of producing a functional amylase according to the invention, preferably a cell capable of over-expressing the amylase according to the invention, for example an Aspergillus strain comprising an increased copy number of a gene or cDNA according to the invention.

In yet another aspect of the invention, a purified polypeptide is provided. The polypeptides according to the invention include the polypeptides encoded by the polynucleotides according to the invention. Especially preferred is a polypeptide having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 or functional equivalents thereof.

Fusion proteins comprising a polypeptide according to the invention are also within the scope of the invention. The invention also provides methods of making the polypeptides according to the invention.

The invention also relates to the use of the amylase according to the invention in any industrial process as described herein

### DETAILED DESCRIPTION OF THE INVENTION

Polynucleotides

The present invention provides polynucleotides encoding an alpha-amylase having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 or functional equivalents thereof. The sequence of the genes encoding a protein according to the invention was determined by sequencing a genomic clone obtained from Aspergillus niger. The invention provides polynucleotide sequences comprising the gene encoding the A niger alpha amylase as well as its complete cDNA sequence and its coding sequence. Accordingly, the invention relates to an isolated polynucleotide comprising a nucleotide sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12 or functional equivalents thereof.

More in particular, the invention relates to an isolated polynucleotide hybridisable under stringent conditions, preferably under highly stringent conditions, to a polynucleotide having a nucleotide sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12. Advantageously, such polynucleotides may be obtained from filamentous fungi, in particular from Aspergillus niger. More specifically, the invention relates to an isolated polynucleotide having a nucleotide sequence having a nucleotide sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12.

The invention also relates to an isolated polynucleotide encoding at least one functional domain of a polypeptide having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 or functional equivalents thereof.

As used herein, the terms “gene” and “recombinant gene” refer to nucleic acid molecules which may be isolated from chromosomal DNA, which include an open reading frame encoding a protein, e.g. an A. niger amylase. A gene may include coding sequences, non-coding sequences, introns and regulatory sequences. Moreover, a gene refers to an isolated nucleic acid molecule as defined herein.

A nucleic acid molecule of the present invention, such as a nucleic acid molecule having a nucleotide sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12 or a functional equivalent thereof, can be isolated using standard molecular biology techniques and the sequence information provided herein. For example, using all or portion of the nucleic acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12 as a hybridization probe, nucleic acid molecules according to the invention can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12 can be isolated by the polymerase chain reaction (PCR) using synthetic oligonucleotide primers designed based upon the sequence information provided herein.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis.

Furthermore, oligonucleotides corresponding to or hybridisable to nucleotide sequences according to the invention can be prepared by standard synthetic techniques, e.g., using an automated DNA synthesizer.

In a preferred embodiment, an isolated nucleic acid molecule of the invention comprises the nucleotide sequence shown in SEQ ID NO: 7, SEQ ID NO: 8, SEQ ID NO: 9, SEQ ID NO: 10, SEQ ID NO: 11 or SEQ ID NO: 12. The sequence information provided in SEQ ID NO: 7, SEQ ID NO: 8, SEQ ID NO: 9, SEQ ID NO: 10, SEQ ID NO: 11 or SEQ ID NO: 12 corresponds to the coding region of the A. niger alpha amylases genes provided in SEQ ID NO: 1, SEQ ID NO: 2, SEQ ID NO: 3, SEQ ID NO: 4, SEQ ID NO: 5 or SEQ ID NO: 6 respectively. This cDNA comprises sequences encoding the A. niger alpha amylases having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 respectively.

In another preferred embodiment, an isolated nucleic acid molecule of the invention comprises a nucleic acid molecule which is a complement of the nucleotide sequence shown in SEQ ID NO: 1, SEQ ID NO: 2, SEQ ID NO: 3, SEQ ID NO: 4, SEQ ID NO: 6, SEQ ID NO: 6, SEQ ID NO: 7, SEQ ID NO: 8, SEQ ID NO: 9, SEQ ID NO: 10, SEQ ID NO: 11 or SEQ ID NO: 12 or a functional equivalent of these nucleotide sequences.

A nucleic acid molecule which is complementary to another nucleotide sequence is one which is sufficiently complementary to the other nucleotide sequence such that it can hybridize to the other nucleotide sequence thereby forming a stable duplex.

One aspect of the invention pertains to isolated nucleic acid molecules that encode a polypeptide of the invention or a functional equivalent thereof such as a biologically active fragment or domain, as well as nucleic acid molecules sufficient for use as hybridisation probes to identify nucleic acid molecules encoding a polypeptide of the invention and fragments of such nucleic acid molecules suitable for use as PCR primers for the amplification or mutation of nucleic acid molecules.

An “isolated polynucleotide” or “isolated nucleic acid” is a DNA or RNA that is not immediately contiguous with both of the coding sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promotor) sequences that are immediately contiguous to the coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant DNA that is part of a hybrid gene encoding an additional polypeptide that is substantially free of cellular material, viral material, or culture medium (when produced by recombinant DNA techniques), or chemical precursors or other chemicals (when chemically synthesized). Moreover, an “isolated nucleic acid fragment” is a nucleic acid fragment that is not naturally occurring as a fragment and would not be found in the natural state.

As used herein, the terms “polynucleotide” or “nucleic acid molecule” are intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. The nucleic acid may be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

Another embodiment of the invention provides an isolated nucleic acid molecule which is antisense to a nucleic acid molecule according to the invention. Also included within the scope of the invention are the complement strands of the nucleic acid molecules described herein.

In certain applications it may be advantageous to have products that are free of amylase activity. Such products may be produced in microorganisms wherein an amylase gene according to the invention is eliminated or wherein its activity is reduced. Such microorganisms may be obtained by recombinant DNA technology, for instance by knocking out the expression of a gene according to the invention. Amylase deficient mutants may be advantageously used for the production of milk clotting enzymes where contamination with amylases is undesired.

Instead of elimination of amylase activities via disruption or mutagenesis, reduced amylase activity can also be achieved via down-regulation of the amylase activities. This may be achieved by genetically altering the promoter or other regulatory sequences of the gene(s) according to the invention. With the help of the sequence information provided herein, the skilled person will know how to achieve the goal of providing mutant microorganisms with reduced or eliminated amylase activity.

Sequencing Errors

The sequence information as provided herein should not be so narrowly construed as to require inclusion of erroneously identified bases. The specific sequences disclosed herein can be readily used to isolate the complete gene from filamentous fungi, in particular A niger which in turn can easily be subjected to further sequence analyses thereby identifying sequencing errors.

Unless otherwise indicated, all nucleotide sequences determined by sequencing a DNA molecule herein were determined using an automated DNA sequencer and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

The person skilled in the art is capable of identifying such erroneously identified bases and knows how to correct for such errors.

Nucleic Acid Fragments, Probes and Primers

A nucleic acid molecule according to the invention may comprise only a portion or a fragment of the nucleic acid sequence shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12, for example a fragment which can be used as a probe or primer or a fragment encoding a portion of a protein according to the invention. The nucleotide sequence determined from the cloning of the alpha amylase gene and cDNA allows for the generation of probes and primers designed for use in identifying and/or cloning other alpha amylase family members, as well as homologues from other species. The probe/primer typically comprises substantially purified oligonucleotide which typically comprises a region of nucleotide sequence that hybridizes preferably under highly stringent conditions to at least about 12 or 15, preferably about 18 or 20, preferably about 22 or 25, more preferably about 30, 35, 40, 45, 50, 55, 60, 65, or 75 or more consecutive nucleotides of a nucleotide sequence shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12 or of a functional equivalent thereof.

Probes based on the nucleotide sequences provided herein can be used to detect transcripts or genomic sequences encoding the same or homologous proteins for instance in other organisms. In preferred embodiments, the probe further comprises a label group attached thereto, e.g., the label group can be a radioisotope, a fluorescent compound, an enzyme, or an enzyme cofactor. Such probes can also be used as part of a diagnostic test kit for identifying cells which express an alpha-amylase.

Identity & Homology

The terms “homology” or “percent identity” are used interchangeably herein. For the purpose of this invention, it is defined here that in order to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid sequence or nucleic add sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Preferably, the two sequences are the same length.

The skilled person will be aware of the fact that several different computer programms are available to determine the homology between two sequences. For instance, a comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (formerly available at http://www.gcg.com now at http://www.accelrys.com), using either a Blossom 62 matrix or a PAM250 matrix, and a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the GCG software package (formerly available at http://www.gcg.com now at http://www.accelrys.com), using a NWSgapdna.CMP matrix and a gap weight of 40, 50, 60, 70, or 80 and a length weight of 1, 2, 3, 4, 5, or 6. In another embodiment, the percent identity two amino acid or nucleotide sequence is determined using the algorithm of E. Meyers and W. Miller (CABIOS, 4:11-17 (1989) which has been incorporated into the ALIGN program (version 2.0) (available at: http://vega.igh.cnrs.fr/bin/align-guess.cgi) using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

Hybridisation

As used herein, the term “hybridizing” is intended to describe conditions for hybridization and washing under which nucleotide sequences at least about 50%, at least about 40%, at least about 70%, more preferably at least about 80%, even more preferably at least about 85% to 90%, more preferably at least 95% homologous to each other typically remain hybridized to each other.

A preferred, non-limiting example of such hybridization conditions are hybridization in 6× sodium chloride/sodium citrate (SSC) at about 45° C., followed by one or more washes in 1×SSC, 0.1% SDS at 50° C., preferably at 55° C., preferably at 60° C. and even more preferably at 65° C.

Highly stringent conditions include, for example, hybridizing at 68° C. in 5×SSC/5× Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridisation conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-standed cDNA clone).

Obtaining Full Length DNA from Other Organisms

In a typical approach, cDNA libraries constructed from other organisms, e.g. filamentous fungi, in particular from the species Aspergillus can be screened.

For example, Aspergillus strains can be screened for homologous polynucleotides by Northern blot analysis. Upon detection of transcripts homologous to polynucleotides according to the invention, cDNA libraries can be constructed from RNA isolated from the appropriate strain, utilizing standard techniques well known to those of skill in the art. Alternatively, a total genomic DNA library can be screened using a probe hybridisable to a polynucleotide according to the invention.

Homologous gene sequences can be isolated, for example, by performing PCR using two degenerate oligonucleotide primer pools designed on the basis of nucleotide sequences as taught herein.

The template for the reaction can be cDNA obtained by reverse transcription of mRNA prepared from strains known or suspected to express a polynucleotide according to the invention. The PCR product can be subcloned and sequenced to ensure that the amplified sequences represent the sequences of a new alpha amylase nucleic acid sequence, or a functional equivalent thereof.

The PCR fragment can then be used to isolate a full length cDNA clone by a variety of known methods. For example, the amplified fragment can be labeled and used to screen a bacteriophage or cosmid cDNA library. Alternatively, the labeled fragment can be used to screen a genomic library.

PCR technology also can be used to isolate full length cDNA sequences from other organisms. For example, RNA can be isolated, following standard procedures, from an appropriate cellular or tissue source. A reverse transcription reaction can be performed on the RNA using an oligonucleotide primer specific for the most 5′ end of the amplified fragment for the priming of first strand synthesis.

The resulting RNA/DNA hybrid can then be “tailed” (e.g., with guanines) using a standard terminal transferase reaction, the hybrid can be digested with RNase H, and second strand synthesis can then be primed (e.g., with a poly-C primer). Thus, cDNA sequences upstream of the amplified fragment can easily be isolated. For a review of useful cloning strategies, see e.g., Sambrook et al., supra; and Ausubel et al., supra.

Vectors

Another aspect of the invention pertains to vectors, preferably expression vectors, containing a nucleic acid encoding a protein according to the invention or a functional equivalent thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. The terms “plasmid” and “vector” can be used interchangeably herein as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vector includes one or more regulatory sequences, selected on the basis of the host cells to be used for expression, which is operatively linked to the nucleic acid sequence to be expressed. Within a recombinant expression vector, “operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signal). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide, sequence only in a certain host cell (e.g. tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, encoded by nucleic acids as described herein (e.g. alpha-amylases, mutant alpha amylases, fragments thereof, variants or functional equivalents thereof, fusion proteins, etc.).

The recombinant expression vectors of the invention can be designed for expression of alpha amylases in prokaryotic or eukaryotic cells. For example, a protein according to the invention can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression vectors useful in the present invention include chromosomal-, episomal- and virus-derived vectors e.g., vectors derived from bacterial plasmids, bacteriophage, yeast episome, yeast chromosomal elements, viruses such as baculoviruses, papova viruses, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled person. In a specific embodiment, promoters are preferred that are capable of directing a high expression level of amylases in filamentous fungi. Such promoters are known in the art. The expression constructs may contain sites for transcription initiation, termination, and, in the transcribed region, a ribosome binding site for translation. The coding portion of the mature transcripts expressed by the constructs will include a translation initiating AUG at the beginning and a termination codon appropriately positioned at the end of the polypeptide to be translated.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-percipitation, DEAE-dextran-mediated transfection, transduction, infection, lipofection, cationic lipidmediated transfection or electroporation. Suitable methods for transforming or transfecting host cells can be found in Sambrook, et al. (Molecular Cloning: A Laboratory Manual, 2^{nd}, ed. Cold Spring Harbor Laboratory, Cold Spring Harbor Laboratory Press, Cold Spring Harbor, N.Y., 1989), Davis et al., Basic Methods in Molecular Biology (1986) and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methatrexate. Nucleic acid encoding a selectable marker can be introduced into a host cell on the same vector as that encoding a protein according to the invention or can be introduced on a separate vector. Cells stably transfected with the introduced nucleic acid can be identified by drug selection (e.g. cells that have incorporated the selectable marker gene will survive, while the other cells die).

Expression of proteins in prokaryotes is often carried out in E. coli with vectors containing constitutive or inducible promoters directing the expression of either fusion or non-fusion proteins. Fusion vectors add a number of amino acids to a protein encoded therein, e.g. to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognation sequences, include Factor Xa, thrombin and enterokinase.

As indicated, the expression vectors will preferably contain selectable markers. Such markers include dihydrofolate reductase or neomycin resistance for eukarotic cell culture and tetracyline or ampicilling resistance for culturing in E. coli and other bacteria. Representative examples of appropriate host include bacterial cells, such as E. coli, Streptomyces and Salmonella typhimurium; fungal cells, such as yeast; insect cells such as Drosophila S2 and Spodoptera Sf9; animal cells such as CHO, COS and Bowes melanoma; and plant cells. Appropriate culture media and conditions for the above-described host cells are known in the art.

Among vectors preferred for use in bacteria are pQE70, pQE60 and PQE-9, available from Qiagen; pBS vectors, Phagescript vectors, Bluescript vectors, pNH8A, pNH16A, pNH18A, pNH46A, available from Stratagene; and ptrc99a, pKK223-3, pKK233-3, pDR540, pRIT5 available from Pharmacia. Among preferred eukaryotic vectors are PWLNEO, pSV2CAT, pOG44, pZT1 and pSG available from Stratagene; and pSVK3, pBPV, pMSG and pSVL available from Pharmacia. Other suitable vectors will be readily apparent to the skilled artisan.

Among known bacterial promoters for use in the present invention include E. coli lacI and lacZ promoters, the T3 and T7 promoters, the gpt promoter, the lambda PR, PL promoters and the trp promoter, the HSV thymidine kinase promoter, the early and late SV40 promoters, the promoters of retroviral LTRs, such as those of the Rous sarcoma virus (“RSV”), and metallothionein promoters, such as the mouse metallothionein-1 promoter.

Transcription of the DNA encoding the polypeptides of the present invention by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at bp 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretation signal may be incorporated into the expressed polypeptide. The signals may be endogenous to the polypeptide or they may be heterologous signals.

The polypeptide may be expressed in a modified form, such as a fusion protein, and may include not only secretion signals but also additional heterologous functional regions. Thus, for instance, a region of additional amino acids, particularly charged amino acids, may be added to the N-terminus of the polypeptide to improve stability and persistence in the host cell, during purification or during subsequent handling and storage. Also, peptide moieties may be added to the polypeptide to facilitate purification.

Polypeptides According to the Invention

The invention provides an isolated polypeptide having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18, an amino acid sequence obtainable by expressing the polynucleotide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12 in an appropriate host. Also, a peptide or polypeptide comprising a functional equivalent of the above polypeptides is comprised within the present invention. The above polypeptides are collectively comprised in the term “polypeptides according to the invention”

The terms “peptide” and “oligopeptide” are considered synonymous (as is commonly recognized) and each term can be used interchangeably as the context requires to indicate a chain of at least two amino acids coupled by peptidyl linkages. The word “polypeptide” is used herein for chains containing more than seven amino acid residues. All oligopeptide and polypeptide formulas or sequences herein are written from left to right and in the direction from amino terminus to carboxy terminus. The one-letter code of amino acids used herein is commonly known in the art and can be found in Sambrook, et al. (Molecular Cloning: A Laboratory Manual, 2^{nd}, ed. Cold Spring Harbor Laboratory, Cold Spring Harbor Laboratory Press, Cold Spring Harbor, N.Y., 1989)

By “isolated” polypeptide or protein is intended a polypeptide or protein removed from its native environment. For example, recombinantly produced polypeptides and proteins expressed in host cells are considered isolated for the purpose of the invention as are native or recombinant polypeptides which have been substantially purified by any suitable technique such as, for example, the single-step purification method disclosed in Smith and Johnson, Gene 67:31-40 (1988).

The amylase according to the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography “HPLC”) is employed for purification.

Polypeptides of the present invention include naturally purified products, products of chemical synthetic procedures, and products produced by recombinant techniques from a prokaryotic or eukaryotic host including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Protein Fragments

The invention also features biologically active fragments of the polypeptides according to the invention.

Biologically active fragments of a polypeptide of the invention include polypeptides comprising amino acid sequences sufficiently identical to or derived from the amino acid sequence of the protein (e.g., the amino acid sequence of SEQ ID NO: 13, SEQ ID NO: 14, SEQ ID NO: 15, SEQ ID NO: 16, SEQ ID NO: 17 or SEQ ID NO: 18), which include fewer amino acids than the full length protein, and exhibit at least one biological activity of the corresponding full-length protein. Typically, biologically active fragments comprise a domain or motif with at least one activity of the alpha-amylase. A biologically active fragment of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the biological activities of the native form of a polypeptide of the invention.

The invention also features nucleic acid fragments which encode the above biologically active fragments of the alpha amylase.

Fusion Proteins

The proteins of the present invention or functional equivalents thereof, e.g., biologically active portions thereof, can be operatively linked to a non-alpha-amylase polypeptide (e.g., heterologous amino acid sequences) to form fusion proteins. As used herein, a “chimeric protein” or “fusion protein” comprises an alpha amylase polypeptide operatively linked to a non-alpha-amylase polypeptide. In a preferred embodiment, a fusion protein comprises at least one biologically active fragment of a protein according to the invention. In this context, the term “operatively linked” is intended to indicate that the alpha amylase and the non-alpha amylase are fused in-frame to each other either to the N-terminus or C-terminus of the alpha amylase.

For example, in one embodiment, the fusion protein is a GST-fusion protein in which the alpha amylase sequences are fused to the C-terminus of the GST sequences. Such fusion proteins can facilitate the purification of recombinant PROTEIN ACCORDING TO THE INVENTION. In another embodiment, the fusion protein comprises a protein according to the invention fused to a heterologous signal sequence at its N-terminus. In certain host cells (e.g., mammalian and Yeast host cells), expression and/or secretion of a protein according to the invention can be increased through use of a hetereologous signal sequence.

In another example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokarytic heterologous signal sequences include the phoA secretory signal (Sambrook et al., supra) and the protein A secretory signal (Pharmacia Biotech; Piscataway, N.J.).

A signal sequence can be used to facilitate secretion and isolation of a protein or polypeptide of the invention. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain. Thus, for instance, the sequence encoding the polypeptide may be fused to a marker sequence, such as a sequence encoding a peptide, which facilitates purification of the fused polypeptide. In certain preferred embodiments of this aspect of the invention, the marker sequence is a hexa-histidine peptide, such as the tag provided in a pQE vector (Qiagen, Inc.), among others, many of which are commercially available. As described in Gentz et al, Proc. Natl. Acad. Sci. USA 86:821-824 (1989), for instance, hexa-histidine provides for convenient purification of the fusion protein. The HA tag is another peptide useful for purification which corresponds to an epitope derived of influenza hemaglutinin protein, which has been described by Wilson et al., Cell 37:767 (1984), for instance.

Preferably, a chimeric or fusion protein comprising a protein according to the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g, a GST polypeptide). A nucleic acid according to the invention can be cloned into such an expression vector such that the fusion moiety is linked in-frame to the fusion moiety in order to express a fusion protein comprising a protein according to the invention.

Functional Equivalents

The terms “functional equivalents” and “functional variants” are used interchangeably herein. Functional equivalents of the alpha amylase encoding DNA fragments described herein are isolated DNA fragments that encode a polypeptide that exhibits a particular function of the A. niger amylase as defined herein. A functional equivalent of a polypeptide according to the invention is a polypeptide that exhibits at least one function of an A. niger amylase as defined herein. Functional equivalents therefore also encompass biologically active fragments.

Functional protein or polypeptide equivalents may contain only conservative substitutions of one or more amino acids in the sequences provided in SEQ ID NO: 13, SEQ ID NO: 14, SEQ ID NO: 15, SEQ ID NO: 16, SEQ ID NO: 17 or SEQ ID NO: 18 or substitutions, insertions or deletions of non-essential amino acids. Accordingly, a non-essential amino acid is a residue that can be altered in SEQ ID NO: 13, SEQ ID NO: 14, SEQ ID NO: 15, SEQ ID NO: 16, SEQ ID NO: 17 or SEQ ID NO: 18 without substantially altering the biological function. For example, amino acid residues that are conserved among the proteins of the present invention, are predicted to be particularly unamenable to alteration. Furthermore, amino acids conserved among the proteins according to the present invention and other amylases are not likely to be amenable to alteration.

The term “conservative substitution” is intended to mean that a substitution in which the amino acid residue is replaced with an amino add residue having a similar side chain. These families are known in the art and include amino acids with basic side chains (e.g., lysine, arginine and hystidine), acidic side chains (e.g. aspartic acid, glutamic acid), uncharged polar side chains (e.g., glycine, asparagines, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine tryptophan, histidine).

Functional nucleic acid equivalents may typically contain silent mutations or mutations that do not alter the biological function of encoded polypeptide. Accordingly, the invention provides nucleic acid molecules encoding proteins that contain changes in amino acid residues that are not essential for a particular biological activity. Such proteins differ in amino acid sequence from SEQ ID NO: 13, SEQ ID NO: 14, SEQ ID NO: 15, SEQ ID NO: 16, SEQ ID NO: 17 or SEQ ID NO: 18 yet retain at least one biological activity. In one embodiment the isolated nucleic acid molecule comprises a nucleotide sequence encoding a protein, wherein the protein comprises a substantially homologous amino acid sequence of at least about 40%, 65%, 70%, 75%, 80%, 85%, 90%, 95%, 96%, 97%, 98%, 99% or more homologous to an amino acid sequence selected from the group consisting of SEQ ID NO: 13, SEQ ID NO: 14, SEQ ID NO: 15, SEQ ID NO: 16, SEQ ID NO: 17 or SEQ ID NO: 18.

For example, guidance concerning how to make phenotypically silent amino acid substitutions is provided in Bowie, J. U. et al., Science 247:1306-1310 (1990) wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As the authors state, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al, supra, and the references cited therein.

An isolated nucleic acid molecule encoding a protein homologous to the protein having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 can be created by introducing one or more nucleotide substitutions, additions or deletions into the coding nucleotide sequences having a nucleotide sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12 such that one or more amino acid substitutions, deletions or insertions are introduced into the encoded protein. Such mutations may be introduced by standard techniques, such as site-directed mutagenesis and PCR-mediated mutagenesis.

The term “functional equivalents” also encompasses orthologues of the A. niger alpha amylases provided herein. Orthologues of the A. niger alpha amylase are proteins that can be isolated from other strains or species and possess a similar or identical biological activity. Such orthologues can readily be identified as comprising an amino acid sequence that is substantially homologous to an amino acid sequence selected from the group consisting of SEQ ID NO: 13, SEQ ID NO: 14, SEQ ID NO: 15, SEQ ID NO: 16, SEQ ID NO: 17 or SEQ ID NO: 18.

As defined herein, the term “substantially homologous” refers to a first amino acid or nucleotide sequence which contains a sufficient or minimum number of identical or equivalent (e.g., with similar side chain) amino acids or nucleotides to a second amino acid or nucleotide sequence such that the first and the second amino acid or nucleotide sequences have a common domain. For example, amino acid or nucleotide sequences which contain a common domain having about 40%, preferably 65%, more preferably 70%, even more preferably 75%, 80%, 85%, 90%, 95%, 96%, 97%, 98% or 99% identity or more are defined herein as sufficiently identical.

Also, nucleic acids encoding other alpha amylase family members, which thus have a nucleotide sequence that differs from a sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12, are within the scope of the invention. Moreover, nucleic acids encoding alpha amylases from different species which thus have a nucleotide sequence which differs from a sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12 are within the scope of the invention.

Nucleic acid molecules corresponding to variants (e.g. natural allelic variants) and homologues of the DNA according to the invention can be isolated based on their homology to the nucleic acids disclosed herein using the cDNAs disclosed herein or a suitable fragment thereof, as a hybridisation probe according to standard hybridisation techniques preferably under highly stringent hybridisation conditions.

In addition to naturally occurring allelic variants of the A niger sequences provided herein, the skilled person will recognise that changes can be introduced by mutation into the nucleotide sequence selected from the group consisting of SEQ ID NO: 1, SEQ ID NO: 2, SEQ ID NO: 3, SEQ ID NO: 4, SEQ ID NO: 5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12 thereby leading to changes in the amino acid sequence of the alpha amylase protein without substantially altering the function of the protein.

In another aspect of the invention, improved alpha amylases are provided. Improved alpha amylases are proteins wherein at least one biological activity is improved. Such proteins may be obtained by randomly introducing mutations along all or part of the coding sequence, such as by saturation mutagenesis, and the resulting mutants can be expressed recombinantly and screened for biological activity. For instance, the art provides for standard assays for measuring the enzymatic activity of amylases and thus improved proteins may easily be selected.

In a preferred embodiment the alpha amylase has an amino acid sequence having an amino acid sequence selected from the group consisting of SEQ ID NO: 13, SEQ ID NO: 14, SEQ ID NO: 15, SEQ ID NO: 16, SEQ ID NO: 17 pr SEQ ID NO: 18. In another embodiment, the alpha amylase is substantially homologous to an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 and retains at least one biological activity of a polypeptide having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18, yet differs in amino acid sequence due to natural variation or mutagenesis as described above.

In a further preferred embodiment, the alpha amylase has an amino acid sequence encoded by an isolated nucleic acid fragment capable of hybridising to a nucleic acid having a nucleotide sequence selected from the group consisting of SEQ ID NO: 1, SEQ ID NO: 2, SEQ ID NO: 3, SEQ ID NO: 4, SEQ ID NO:5 or SEQ ID NO: 6 or from the group consisting of SEQ ID NO: 7, SEQ ID NO: 8, SEQ ID NO: 9, SEQ ID NO: 10, SEQ ID NO: 11 or SEQ ID NO: 12, preferably under highly stringent hybridisation conditions.

Accordingly, an alpha amylase according to the invention is an isolated protein which comprises an amino acid sequence at least about 40%, 65%, 70%, 75%, 80%, 85%, 90%, 95%, 96%, 97%, 98%, 99% or more homologous to an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 and retains at least one functional activity of the polypeptide having an amino acid sequence selected from the group consisting of SEQ ID NO: 13, SEQ ID NO: 14, SEQ ID NO: 15, SEQ ID NO: 16, SEQ ID NO:17 or SEQ ID NO: 18.

Functional equivalents of a protein according to the invention can also be identified e.g. by screening combinatorial libraries of mutants, e.g. truncation mutants, of the protein of the invention for amylase activity. In one embodiment, a variegated library of variants is generated by combinatorial mutagenesis at the nucleic acid level. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods that can be used to produce libraries of potential variants of the polypeptides of the invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequence of a polypeptide of the invention can be used to generate a variegated population of polypeptides for screening a subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations of truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

It will be apparent for the person skilled in the art that DNA sequence polymorphisms may exist that may lead to changes in the amino acid sequence of the alpha amylase within a given population. Such genetic polymorphisms may exist in cells from different populations or within a population due to natural allelic variation. Allelic variants may also include functional equivalents.

Fragments of a polynucleotide according to the invention may also comprise polynucleotides not encoding functional polypeptides. Such polynucleotides may function as probes or primers for a PCR reaction.

Nucleic acids according to the invention irrespective of whether they encode functional or non-functional polypeptides, can be used as hybridization probes or polymerase chain reaction (PCR) primers. Uses of the nucleic acid molecules of the present invention that do not encode a polypeptide having alpha amylase activity include, inter alia, (1) isolating the gene encoding the alpha amylase, or allelic variants thereof from a cDNA library e.g. from other organisms than A niger; (2) in situ hybridization (e.g. FISH) to metaphase chromosomal spreads to provide precise chromosomal location of the alpha amylase gene as described in Verma et al., Human Chromosomes: a Manual of Basic Techniques, Pergamon Press, New York (1988); (3) Northern blot analysis for detecting expression of alpha amylase mRNA in specific tissues and/or cells and 4) probes and primers that can be used as a diagnostic tool to analyse the presence of a nucleic acid hybridisable to the alpha amylase probe in a given biological (e.g. tissue) sample.

Also encompassed by the invention is a method of obtaining a functional equivalent of an alpha amylase gene or cDNA. Such a method entails obtaining a labelled probe that includes an isolated nucleic acid which encodes all or a portion of the sequence having an amino acid sequence selected from the group consisting of SEQ ID NO: 13, SEQ ID NO: 14, SEQ ID NO: 15, SEQ ID NO: 16, SEQ ID NO: 17 or SEQ ID NO: 18 or a variant thereof; screening a nucleic acid fragment library with the labelled probe under conditions that allow hybridisation of the probe to nucleic acid fragments in the library, thereby forming nucleic acid duplexes, and preparing a full-length gene sequence from the nucleic acid fragments in any labelled duplex to obtain a gene related to the alpha amylase gene.

In one embodiment, a nucleic acid according to the invention is at least 40%, 65%, 70%, 75%, 80%, 85%, 90%, 91%, 92%, 93%, 94%, 95%, 96%, 97%, 98%, 99%, or more homologous to a nucleic acid sequence shown in SEQ ID NO: 1, SEQ ID NO: 2, SEQ ID NO: 3, SEQ ID NO: 4, SEQ ID NO: 5 SEQ ID NO: 6, SEQ ID NO: 7, SEQ ID NO: 8, SEQ ID NO: 9, SEQ ID NO: 10, SEQ ID NO: 11 or SEQ ID NO: 12 or the complement thereof.

In another preferred embodiment a polypeptide of the invention is at least 40%, 65%, 70%, 75%, 80%, 85%, 90%, 91%, 92%, 93%, 94%, 95%, 96%, 97%, 98%, 99%, or more homologous to the amino acid sequence shown in SEQ ID NO: 13, SEQ ID NO: 14, SEQ ID NO: 15, SEQ ID NO: 16, SEQ ID NO: 17 or SEQ ID NO: 18.

Host Cells

In another embodiment, the invention features cells, e.g., transformed host cells or recombinant host cells that contain a nucleic acid encompassed by the invention. A “transformed cell” or “recombinant cell” is a cell into which (or into an ancestor of which) has been introduced, by means of recombinant DNA techniques, a nucleic acid according to the invention. Both prokaryotic and eukaryotic cells are included, e.g., bacteria, fungi, yeast, and the like, especially preferred are cells from filamentous fungi, in particular Aspergillus niger.

A host cell can be chosen that modulates the expression of the inserted sequences, or modifies and processes the gene product in a specific, desired fashion. Such modifications (e.g., glycosylation) and processing (e.g., cleavage) of protein products may facilitate optimal functioning of the protein.

Various host cells have characteristic and specific mechanisms for post-translational processing and modification of proteins and gene products. Appropriate cell lines or host systems familiar to those of skill in the art of molecular biology and/or microbiology can be chosen to ensure the desired and correct modification and processing of the foreign protein expressed. To this end, eukaryotic host cells that possess the cellular machinery for proper processing of the primary transcript, glycosylation, and phosphorylation of the gene product can be used. Such host cells are well known in the art.

Host cells also include, but are not limited to, mammalian cell lines such as CHO, VERO, BHK, HeLa, COS, MDCK, 293, 3T3, W138, and choroid plexus cell lines.

If desired, the polypeptides according to the invention can be produced by a stably-transfected cell line. A number of vectors suitable for stable transfection of mammalian cells are available to the public, methods for constructing such cell lines are also publicly known, e.g., in Ausubel et al. (supra).

Antibodies

The invention further features antibodies, such as monoclonal or polyclonal antibodies, that specifically bind alpha amylases according to the invention.

As used herein, the term “antibody” (Ab) or “monoclonal antibody” (Mab) is meant to include intact molecules as well as antibody fragments (such as, for example, Fab and F(ab′)₂ fragments) which are capable of specifically binding to a protein according to the invention. Fab and F(ab′)₂ fragments lack the Fc fragment of intact antibody, dear more rapidly from the circulation, and may have less non-specific tissue binding of an intact antibody (Wahl et al., J. Nucl. Med. 24:316-325 (1983)). Thus, these fragments are preferred.

The antibodies of the present invention may be prepared by any of a variety of methods. For example, cells expressing the alpha amylase according to the invention or an antigenic fragment thereof can be administered to an animal in order to induce the production of sera containing polyclonal antibodies. In a preferred method, a preparation of a protein according to the invention is prepared and purified to render it substantially free of natural contaminants. Such a preparation is then introduced into an animal in order to produce polyclonal antisera of greater specific activity. In the most preferred method, the antibodies of the present invention are monoclonal antibodies (or alpha amylase-binding fragments thereof). Such monoclonal antibodies can be prepared using hybridoma technology (Kohler et al., Nature 256:495 (1975); Kohler et al., Eur. J. Immunol. 6:511 (1976); Hammerling et al., In: Monoclonal Antibodies and T-Cell Hybridomas, Elsevier, N.Y., pp. 563-681 (1981)). In general, such procedures involve immunizing an animal (preferably a mouse) with a protein according to the invention or, with a cell expressing a protein according to the invention. The splenocytes of thus immunised mice are extracted and fused with a suitable myeloma cell line. Any suitable myeloma cell line may be employed in accordance with the present inventoin; however, it is preferably to employ the parent myeloma cell line (SP₂O), available from the American Type Culture Collection, Rockville, Md. After fusion, the resulting hybridoma cells are selectively maintained in HAT medium, and then cloned by limiting dilution as described by Wands et al. (Gastro-enterology 80:225-232 (1981)). The hybridoma cells obtained through such a selection are then assayed to identify clones which secrete antibodies capable of binding the alpha amylase antigen. In general, the polypeptides can be coupled to a carrier protein, such as KLH, as described in Ausubel et al., supra, mixed with an adjuvant, and injected into a host mammal.

In particular, various host animals can be immunized by injection of a polypeptide of interest Examples of suitable host animals include rabbits, mice, guinea pigs, and rats. Various adjuvants can be used to increase the immunological response, depending on the host species, including but not limited to Freund's (complete and incomplete), adjuvant mineral gels such as aluminum hydroxide, surface actve substances such as lysolecithin, pluronic polyols, polyanions, peptides, oil emulsions, keyhole limpet hemocyanin, dinitrophenol, BCG (bacille Calmette-Guerin) and Corynebacterium parvum. Polyclonal antibodies are heterogeneous populations of antibody molecules derived from the sera of the immunized animals.

Such antibodies can be of any immunoglobulin class including IgG, IgM, IgE, IgA, IgD, and any subclass thereof. The hybridomas producing the mAbs of this invention can be cultivated in vitro or in vivo.

Once produced, polyclonal or monoclonal antibodies are tested for specific recognition of a protein according to the invention or functional equivalent thereof in an immunoassay, such as a Western blot or immunoprecipitation analysis using standard techniques, e.g., as described in Ausubel et al., supra. Antibodies that specifically bind to a protein according to the invention or functional equivalents thereof are useful in the invention. For example, such antibodies can be used in an immunoassay to detect a protein according to the invention in pathogenic or non-pathogenic strains of Aspergillus (e.g., in Aspergillus extracts).

Preferably, antibodies of the invention are produced using fragments of a protein according to the invention that appears likely to be antigenic, by criteria such as high frequency of charged residues. For example, such fragments may be generated by standard techniques of PCR, and then cloned into the pGEX expression vector (Ausubel et al., supra). Fusion proteins may then be expressed in E. coli and purified using a glutathione agarose affinity matrix as described in Ausubel, et al., supra. If desired, several (e.g., two or three) fusions can be generated for each protein, and each fusion can be injected into at least two rabbits. Antisera can be raised by injections in a series, typically including at least three booster injections. Typically, the antisera are checked for their ability to immunoprecipitate a recombinant alpha amylase according to the invention or functional equivalents thereof whereas unrelated proteins may serve as a control for the specificity of the immune reaction.

Alternatively, techniques decribed for the production of single chain antibodies (U.S. Pat. Nos. 4,946,778 and 4,704,692) can be adapted to produce single chain antibodies against a protein according to the invention or functional equivalents thereof. Kits for generating and screening phage display libraries are commercially available, e.g. from Pharmacia.

Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 20791; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246;1275-1281; Griffiths et al. (1993) EMBO J. 12:725-734.

Polyclonal and monoclonal antibodies that specifically bind a protein according to the invention or functional equivalents thereof can be used, for example, to detect expression of gene encoding a protein according to the invention or a functional equivalent thereof e.g. in another strain of Aspergillus. For example, a protein according to the invention can be readily detected in conventional immunoassays of Aspergillus cells or extracts. Examples of suitable assays include, without limitation, Western blotting, ELISAs, radioimmune assays, and the like.

By “specifically binds is meant that an antibody recognizes and binds a particular antigen, e.g., a protein according to the invention polypeptide, but does not substantially recognize and bind other unrelated molecules in a sample.

Antibodies can be purified, for example, by affinity chromatography methods in which the polypeptide antigen is immobilized on a resin.

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in cells or tissue as part of a clinical testing procedure, e.g., to, for example, determine the efficacy of a given treatment regimen or in the diagnosis of Aspergillosis.

Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive materials include ¹²⁵I, ¹³¹I, ³⁵S or ³H.

Preferred epitopes encompassed by the antigenic peptide are regions that are located on the surface of the protein, e.g. hydrophilic regions. Hydrophobicity plots of the proteins of the invention can be used to identify hydrophilic regions.

The antigenic peptide of a protein of the invention comprises at least 7 (preferably 10, 15, 20, or 30) contiguous amino acid residues of an amino acid sequense selected from the group consisting of SEQ ID NO: 13, SEQ ID NO: 14, SEQ ID NO: 15, SEQ ID NO: 16, SEQ ID NO: 17 or SEQ ID NO: 18 and encompasses an epitope of the protein such that an antibody raised against the peptide forms a specific immune complex with the protein.

Preferred epitopes encompassed by the antigenic peptide are regions of a protein according to the invention that are located on the surface of the protein, e.g., hydrophilic regions, hydrophobic regions, alpha regions, beta regions, coil regions, turn regions and flexible regions.

Immunoassays

Qualitative or quantitative determination of a polypeptide according to the present invention in a biological sample can occur using any art-known method. Antibody-based techniques provide special advantages for assaying specific polypeptide levels in a biological sample.

In these, the specific recognition is provided by the primary antibody (polyclonal or monoclonal) but the secondary detection system can utilize fluorescent, enzyme, or other conjugated secondary antibodies. As a result, an immunocomplex is obtained.

Accordingly, the invention provides a method for diagnosing whether a certain organism is infected with Aspergillus comprising the steps of:
Isolating a biological sample from said organism suspected to be infected with Aspergillus,
reacting said biological sample with an antibody according to the invention,
determining whether immune complexes are formed.

Tissues can also be extracted, e.g., with urea and neutral detergent, for the liberation of protein for Western-blot or dot/slot assay. This technique can also be applied to body fluids.

Other antibody-based methods useful for detecting a protein according to the invention, include immunoassays, such as the enzyme linked immunosorbent assay (ELISA) and the radioimmunoassay (RIA). For example, specific monoclonal antibodies against a protein according to the invention can be used both as an immunoabsorbent and as an enzyme-labeled probe to detect and quantify a protein according to the invention. The amount of specific protein present in the sample can be calculated by reference to the amount present in a standard preparation using a linear regression computer algorithm. In another ELISA assay, two distinct specific monoclonal antibodies can be used to detect a protein according to the invention in a biological fluid. In this assay, one of the antibodies is used as the immuno-absorbent and the other as the enzyme-labeled probe.

The above techniques may be conducted essentially as a “one-step” or “two-step” assay. The “one-step” assay involves contacting a protein according to the invention with immobilized antibody and, without washing, contacting the mixture with the labeled antibody. The “two-step” assay involves washing before contacting the mixture with the labeled antibody. Other conventional methods may also be employed as suitable. It is usually desirable to immobilize one component of the assay system on a support, thereby allowing other components of the system to be brought into contact with the component and readily removed from the sample.

Suitable enzyme labels include, for example, those from the oxidase group, which catalyze the production of hydrogen peroxide by reacting with substrate. Activity of an oxidase label may be assayed by measuring the concentration of hydrogen peroxide formed by the enzyme-labelled antibody/substrate reaction.

Besides enzymes, other suitable labels include radioisotopes, such as iodine (¹²¹I, ¹²¹I), carbon (¹⁴C), sulphur (³⁵S), tritium (³H), indium (¹¹²In), and technetium (^{99m}Tc), and fluorescent labels, such as fluorescein and rhodamine, and biotin.

Specific binding of a test compound to a protein according to the invention can be detected, for example, in vitro by reversibly or irreversibly immobilizing a protein according to the invention polypeptide on a substrate, e.g., the surface of a well of a 96-well polystyrene microtitre plate. Methods for immobilizing polypeptides and other small molecules are well known in the art. For example, the microtitre plates can be coated with a protein according to the invention by adding the polypeptide in a solution (typically, at a concentration of 0.05 to 1 mg/ml in a volume of 1-100 ul) to each well, and incubating the plates at room temperature to 37° C. for 0.1 to 36 hours. Polypeptides that are not bound to the plate can be removed by shaking the excess solution from the plate, and then washing the plate (once or repeatedly) with water or a buffer. Typically, the polypeptide is contained in water or a buffer. The plate is then washed with a buffer that lacks the bound polypeptide. To block the free protein-binding sites on the plates, the plates are blocked with a protein that is unrelated to the bound polypeptide. For example, 300 ul of bovine serum albumin (BSA) at a concentration of 2 mg/ml in Tris-HCl is suitable. Suitable substrates include those substrates that contain a defined cross-linking chemistry (e.g., plastic substrates, such as polystyrene, styrene, or polypropylene substrates from Corning Costar Corp. (Cambridge, Mass.), for example). If desired, a beaded particle, e.g., beaded agarose or beaded sepharose, can be used as the substrate.

Binding of the test compound to the polypeptides according to the invention can be detected by any of a variety of art known methods. For example, a specific antibody can be used in an immunoassay. If desired, the antibody can be labeled (e.g., fluorescently or with a radioisotope) and detected directly (see, e.g., West and McMahon, J. Cell Biol. 74:264, 1977). Alternatively, a second antibody can be used for detection (e.g., a labeled antibody that binds the Fc portion of an anti-AN97 antibody). In an alternative detection method, a protein according to the invention is labelled (e.g., with a radioisotope, fluorophore, chromophore, or the like), and the label is detected. In still another method, a protein according to the invention is produced as a fusion protein with a protein that can be detected optically, e.g., green fluorescent protein (which can be detected under UV light). In an alternative method, a protein according to the invention polypeptide can be covalently attached to or fused with an enzyme having a detectable enzymatic activity, such as horse radish peroxidase, alkaline phosphatase, a-galactosidase, or glucose oxidase. Genes encoding all of these enzymes have been cloned and are readily available for use by those of skill in the art. If desired, the fusion protein can include an antigen, and such an antigen can be detected and measured with a polyclonal or monoclonal antibody using conventional methods. Suitable antigens include enzymes (e.g., horse radish peroxidase, alkaline phosphatase, and a-galactosidase) and non-enzymatic polypeptides (e.g., serum proteins, such as BSA and globulins, and milk proteins, such as caseins).

Epitopes, Antigens and Immunogens.

In another aspect, the invention provides a peptide or polypeptide comprising an epitope-bearing portion of a polypeptide of the invention. The epitope of this polypeptide portion is an immunogenic or antigenic epitope of a polypeptide of the invention. An immunogenic epitope” is defined as a part of a protein that elicits an antibody response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes. See, for instance, Geysen, H. M. et al., Proc. Natl. Acad. Sci. USA 81:3998-4002 (1984).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G. et al., Science 219:660-666 (1984). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of six or fewer residues generally are ineffective at inducing antibodies that bind to the mimicked protein; longer, soluble peptides, especially those containing proline residues, usually are effective. Sutcliffe et al., supra, at 661. For instance, 18 of 20 peptides designed according to these guidelines, containing 8-39 residues covering 75% of the sequence of the influenza virus hemagglutinin HAI polypeptide chain, induced antibodies that reacted with the HA1 protein or intact virus; and 12/12 peptides from the MuLV polymerase and 18/18 from the rabies glycoprotein induced antibodies that precipitated the respective proteins.

Antigenic epitope-bearing peptides and polypeptides of the invention are therefore useful to raise antibodies, including monoclonal antibodies, that bind specifically to a polypeptide of the invention. Thus, a high proportion of hybridomas obtained by fusion of spleen cells from donors immunized with an antigen epitope-bearing peptide generally secrete antibody reactive with the native protein. Sutcliffe et al., supra, at 663. The antibodies raised by antigenic epitope bearing peptides or polypeptides are useful to detect the mimicked protein, and antibodies to different peptides may be used for tracking the fate of various regions of a protein precursor which undergoes posttranslation processing. The peptides and antipeptide antibodies may be used in a variety of qualitative or quantitative assays for the mimicked protein, for instance in competition assays since it has been shown that even short peptides (e.g., about 9 amino acids) can bind and displace the larger peptides in immunoprecipitation assays. See, for instance, Wilson, I. A. et al., Cell 37:767-778 at 777 (1984). The antipeptide antibodies of the invention also are useful for purification of the mimicked protein, for instance, by adsorption chromatography using methods well known in the art.

Antigenic epitope-bearing peptides and polypeptides of the invention designed according to the above guidelines preferably contain a sequence of at least seven, more preferably at least nine and most preferably between about 15 to about 30 amino acids contained within the amino acid sequence of a polypeptide of the invention. However, peptides or polypeptides comprising a larger portion of an amino acid sequence of a polypeptide of the invention, containing about 30 to about 50 amino acids, or any length up to and including the entire amino acid sequence of a polypeptide of the invention, also are considered epitope-bearing peptides or polypeptides of the invention and also are useful for inducing antibodies that react with the mimicked protein. Preferably, the amino acid sequence of the epitope-bearing peptide is selected to provide substantial solubility in aqueous solvents (i.e., the sequence includes relatively hydrophilic residues and highly hydrophobic sequences are preferably avoided); and sequences containing proline residues are particularly preferred.

The epitope-bearing peptides and polypeptides of the invention may be produced by any conventional means for making peptides or polypeptides including recombinant means using nucleic acid molecules of the invention. For instance, a short epitope-bearing amino acid sequence may be fused to a larger polypeptide which acts as a carrier during recombinant production and purification, as well as during immunization to produce anti-peptide antibodies.

Epitope-bearing peptides also may be synthesized using known methods of chemical synthesis. For instance, Houghten has described a simple method for synthesis of large numbers of peptides, such as 10-20 mg of 248 different 13 residue peptides representing single amino acid variants of a segment of the HAI polypeptide which were prepared and characterized (by ELISA-type binding studies) in less than four weeks. Houghten, R. A., Proc. Natl. Acad. Sci. USA 82:5131-5135 (1985). This “Simultaneous Multiple Peptide Synthesis (SMPS)” process is further described in U.S. Pat. No. 4,631,211 to Houghten et al. (1986). In this procedure the individual resins for the solid-phase synthesis of various peptides are contained in separate solvent-permeable packets, enabling the optimal use of the many identical repetitive steps involved in solid-phase methods.

A completely manual procedure allows 500-1000 or more syntheses to be conducted simultaneously. Houghten et al., supra, at 5134.

Epitope-bearing peptides and polypeptides of the invention are used to induce antibodies according to methods well known in the art. See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985).

Generally, animals may be immunized with free peptide; however, anti-peptide antibody titer may be boosted by coupling of the peptide to a macromolecular carrier, such as keyhole limpet hemocyanin (KLH) or tetanus toxoid. For instance, peptides containing cysteine may be coupled to carrier using a linker such as maleimidobenzoyl-N-hydroxysuccinimide ester (MBS), while other peptides may be coupled to carrier using a more general linking agent such as glutaraldehyde.

Animals such as rabbits, rats and mice are immunized with either free or carrier coupled peptides, for instance, by intraperitoneal and/or intradermal injection of emulsions containing about 100 ug peptide or carrier protein and Freund's adjuvant. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

Immunogenic epitope-bearing peptides of the invention, i.e., those parts of a protein that elicit an antibody response when the whole protein is the immunogen, are identified according to methods known in the art. For instance, Geysen et al., 1984, supra, discloses a procedure for rapid concurrent synthesis on solid supports of hundreds of peptides of sufficient purity to react in an enzyme-linked immunosorbent assay. Interaction of synthesized peptides with antibodies is then easily detected without removing them from the support. In this manner a peptide bearing an immunogenic epitope of a desired protein may be identified routinely by one of ordinary skill in the art. For instance, the immunologically important epitope in the coat protein of foot-and-mouth disease virus was located by Geysen et al. with a resolution of seven amino acids by synthesis of an overlapping set of all 208 possible hexapeptides covering the entire 213 amino acid sequence of the protein. Then, a complete replacement set of peptides in which all 20 amino acids were substituted in turn at every position within the epitope were synthesized, and the particular amino acids conferring specificity for the reaction with antibody were determined. Thus, peptide analogs of the epitope-bearing peptides of the invention can be made routinely by this method. U.S. Pat. No. 4,708,781 to Geysen (1987) further describes this method of identifying a peptide bearing an immunogenic epitope of a desired protein.

Further still, U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is a topographical equivalent of a ligand which is complementary to the ligand binding site of a particular receptor of interest. Similarly, U.S. Pat. No. 5,480,971 to Houghten, R. A et al. (1996) on Peralkylated Oligopeptide Mixtures discloses linear C₁-C₇-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

Use of Alpha Amylases in Industrial Processes

The invention also relates to the use of a protein according to the invention in a selected number of industrial and pharmaceutical processes. Despite the long term experience obtained with these processes, the amylase according to the invention features a number of significant advantages over the enzymes currently used. Depending on the specific application, these advantages can include aspects like lower production costs, higher specificity towards the substrate, less antigenic, less undesirable side activities, higher yields when produced in a suitable microorganism, more suitable pH and temperature ranges, better tastes of the final product as well as food grade and kosher aspects.

An important aspect of the amylases according to the invention is that they cover a whole range of pH and temperature optima which are ideally suited for a variety of applications. For example many large scale processes benefit from relatively high processing temperatures of 50 degrees C. or higher, e.g. to control the risks of microbial infections. Several alpha amylases according to the invention comply with this demand but at the same time they are not that heat stable that they resist attempts to inactivate the enzyme by an additional heat treatment. The latter feature allows production routes that yield final products free of residual enzyme activity. Similarly many feed and food products have slightly acidic pH values so that amylases with acidic or near neutral pH optima are preferred for their processing. An alpha amylase according to the invention complies with this requirement as well.

1-22. (canceled)

23. An isolated polypeptide that has amylase activity and has an amino acid sequence at least 40% homologous to a sequence selected from the group consisting of SEQ ID NO: 13, SEQ ID NO: 14, SEQ ID NO: 15, SEQ ID NO: 16, SEQ ID NO: 17 and SEQ ID NO: 18.

24. The isolated polypeptide of claim 23 which has an amino acid sequence at least 70% homologous to a sequence selected from the group consisting of SEQ ID NO: 13, SEQ ID NO: 14, SEQ ID NO: 15, SEQ ID NO: 16, SEQ ID NO: 17 and SEQ ID NO: 18.

25. The isolated polypeptide of claim 23 which has an amino acid sequence at least 80% homologous to a sequence selected from the group consisting of SEQ ID NO: 13, SEQ ID NO: 14, SEQ ID NO: 15, SEQ ID NO: 16, SEQ ID NO: 17 and SEQ ID NO: 18.

26. The isolated polypeptide of claim 23 which has an amino acid sequence at least 90% homologous to a sequence selected from the group consisting of SEQ ID NO: 13, SEQ ID NO: 14, SEQ ID NO: 15, SEQ ID NO: 16, SEQ ID NO: 17 and SEQ ID NO: 18.

27. The isolated polypeptide of claim 23 which has an amino acid sequence at least 95% homologous to a sequence selected from the group consisting of SEQ ID NO: 13, SEQ ID NO: 14, SEQ ID NO: 15, SEQ ID NO: 16, SEQ ID NO: 17 and SEQ ID NO: 18.

28. The polypeptide of claim 23 that has the sequence of SEQ ID NO: 13, SEQ ID NO: 14, SEQ ID NO: 15, SEQ ID NO: 16, SEQ ID NO: 17 or SEQ ID NO: 18.

29. The polypeptide of claim 23 obtainable from Aspergillus niger.

30. An isolated polynucleotide encoding at least one functional domain of the polypeptide of claim 23.

31. An isolated polynucleotide encoding at least one functional domain of the polypeptide of claim 28.

32. An isolated polynucleotide encoding the polypeptide of claim 23.

33. An isolated polynucleotide encoding the polypeptide of claim 24.

34. An isolated polynucleotide encoding the polypeptide of claim 25.

35. An isolated polynucleotide encoding the polypeptide of claim 26.

36. An isolated polynucleotide encoding the polypeptide of claim 27.

37. An isolated polynucleotide encoding the polypeptide of claim 28.

38. The polynucleotide of claim 30 hybridizable under high stringency conditions to a polynucleotide selected from the group consisting of SEQ ID NO: 1, SEQ ID NO: 2, SEQ ID NO: 3, SEQ ID NO: 4, SEQ ID NO: 5 SEQ ID NO: 6 or from the group consisting of SEQ ID NO: 7, SEQ ID NO: 8, SEQ ID NO: 9, SEQ ID NO: 10, SEQ ID NO: 11 and SEQ ID NO: 12 and their complements.

39. The polynucleotide of claim 32 hybridizable under high stringency conditions to a polynucleotide selected from the group consisting of SEQ ID NO: 1, SEQ ID NO: 2, SEQ ID NO: 3, SEQ ID NO: 4, SEQ ID NO: 5 and SEQ ID NO: 6 or from the group consisting of SEQ ID NO: 7, SEQ ID NO: 8, SEQ ID NO: 9, SEQ ID NO: 10, SEQ ID NO: 1 and SEQ ID NO: 12 and their complements.

40. The polynucleotide of claim 39 selected from the group consisting of SEQ ID NO: 1, SEQ ID NO: 2, SEQ ID NO: 3, SEQ ID NO: 4, SEQ ID NO: 5 and SEQ ID NO: 6 or from the group consisting of SEQ ID NO: 7, SEQ ID NO: 8, SEQ ID NO: 9, SEQ ID NO: 10, SEQ ID NO: 11 and SEQ ID NO: 12 and their complements.

41. The polynucleotide of claim 39 obtainable from a filamentous fungus.

42. The polynucleotide of claim 41 obtainable from A. niger.

43. A vector comprising a polynucleotide sequence of claim 30.

44. A vector comprising a polynucleotide sequence of claim 32.

45. A recombinant nucleic acid molecule wherein the polynucleotide sequence of claim 30 is operatively linked with regulatory sequences suitable for expression of said polynucleotide sequence in a suitable host cell.

46. A recombinant nucleic acid molecule wherein the polynucleotide sequence of claim 32 is operatively linked with regulatory sequences suitable for expression of said polynucleotide sequence in a suitable host cell.

47. A recombinant host cell comprising the nucleic acid molecule of claim 45.

48. A recombinant host cell comprising the nucleic acid molecule of claim 46.

49. A method for manufacturing an amylase comprising culturing said cell of claim 47 under conditions allowing expression of said polynucleotide sequence and optionally purifying the encoded amylase from said cell or culture medium.

50. A method for manufacturing an amylase comprising culturing said cell of claim 48 under conditions allowing expression of said polynucleotide sequence and optionally purifying the encoded amylase from said cell or culture medium.

51. A recombinant host cell expressing a polypeptide of claim 23.

52. A recombinant host cell expressing a polypeptide of claim 28.

53. Purified antibodies reactive with the polypeptide of claim 23.

54. Purified antibodies reactive with the polypeptide of claim 28.

55. A fusion protein comprising the polypeptide of claim 23.

56. A fusion protein comprising the polypeptide of claim 28.

57. Recombinant amylase comprising a functional domain of the polypeptide of claim 23.

58. Recombinant amylase comprising a functional domain of the polypeptide of claim 28.

59. An isolated polynucleotide, comprising at least 12 consecutive nucleotides of a nucleotide sequence that hybridizes under stringent conditions to a sequence selected from the group consisting of SEQ ID NO: 1, SEQ ID NO: 2, SEQ ID NO: 3, SEQ ID NO: 4, SEQ ID NO: 5 and SEQ ID NO: 6 or from the group consisting of SEQ ID NO: 7, SEQ ID NO: 8, SEQ ID NO: 9, SEQ ID NO: 10, SEQ ID NO: 1 and SEQ ID NO: 12, and their complements.

60. The isolated polynucleotide of claim 59 comprising at least 12 consecutive nucleotides of a sequence selected from the group consisting of SEQ ID NO: 1, SEQ ID NO: 2, SEQ ID NO: 3, SEQ ID NO: 4, SEQ ID NO: 5 and SEQ ID NO: 6 or from the group consisting of SEQ ID NO: 7, SEQ ID NO: 8, SEQ ID NO: 9, SEQ ID NO: 10, SEQ ID NO: 11 and SEQ ID NO: 12, and their complements.

61. A method for manufacturing a polynucleotide according to claim 30 comprising culturing a host cell transformed with said polynucleotide and isolating said polynucleotide from said host cell.

62. A method for manufacturing a polynucleotide according to claim 32 comprising culturing a host cell transformed with said polynucleotide and isolating said polynucleotide from said host cell.
